(19) United States
(12) Patent Application Publication
(10) Pub. No.: US 2026/0347807 A1
(43) Pub. Date: Nov. 5, 2026

(54) METHOD FOR PRODUCING A MICROORGANISM HAVING N-DEACETYLATION ACTIVITY AND N-SULFATION ACTIVITY, METHOD FOR PRODUCING A HEPAROSAN-DERIVED COMPOUND, AND A MICROORGANISM HAVING N-DEACETYLATION ACTIVITY AND N-SULFATION ACTIVITY

(71) Applicants: RENNSELAER POLYTECHNIC INSTITUTE, Troy, NY (US); OTSUKA PHARMACEUTICAL FACTORY, INC., Naruto-shi, Tokushima (JP); KIRIN BIOMATERIALS CO., LTD., Tokyo (JP)

(72) Inventors: Marc DOUAISI, Troy, NY (US); Jiaqi HUANG, Troy, NY (US); Elena PASKALEVA, Troy, NY (US); Matthew BRIER, Troy, NY (US); Robert J. LINHARDT, Troy, NY (US); Jonathan S. DORDICK, Troy, NY (US); Asako KIKUCHI, Tokyo (JP); Yukimi FUKUDA, Tokyo (JP); Tetsuro UJIHARA, Tokyo (JP)

(73) Assignees: RENNSELAER POLYTECHNIC INSTITUTE, Troy, NY (US); OTSUKA PHARMACEUTICAL FACTORY, INC., Naruto-shi, Tokushima (JP); KIRIN BIOMATERIALS CO., LTD., Tokyo (JP)

(21) Appl. No.: 19/488,400

(22) PCT Filed: May 27, 2024
(86) PCT No.: PCT/JP2024/019404

§ 371 Date: Nov. 26, 2025

Related U.S. Application Data
(63) Continuation of application No. 18/326,155, filed on May 31, 2023, now abandoned.

(51) Int. Cl. C12N 9/10; C12N 15/52; C12P 19/26

(52) U.S. Cl. CPC C12N 9/13; C12N 15/52; C12P 19/26; C12Y 208/02008; C12Y 208/02023; C12Y 208/02029

(57) ABSTRACT

The present invention relates to a method for producing a microorganism having N-deacetylation activity and N-sulfation activity, including a step (I) of modifying a nucleotide sequence of a DNA encoding N-deacetylase/N-sulfotransferase, and a step (II) of introducing the DNA containing the nucleotide sequence modified in the step (I) into a microorganism in an expressible manner, wherein the step (I) is a step of performing codon optimization according to the codon usage frequency of a biological species different from the microorganism. Further, the invention relates to a method for producing a microorganism having N-deacetylation activity and N-sulfation activity, including a step (i) of expressing a specific protein in a microorganism.

### TECHNICAL FIELD

The present invention relates to a method for producing a microorganism having N-deacetylation activity and N-sulfation activity, a method for producing a heparosan-derived compound, and a microorganism having N-deacetylation activity and N-sulfation activity.

### BACKGROUND ART

Heparin, a sulfated polysaccharide, is an anticoagulant agent used for treating thromboembolism and disseminated intravascular coagulation syndrome, preventing coagulation during artificial dialysis and in extracorporeal circulation, and the like. Industrially, heparin is extracted and purified from animal organs, mainly from the intestinal mucosa of pigs.

Since the 2008 heparin adulteration event that caused numerous fatalities due to contamination of pig-derived heparin with impurities such as oversulfated chondroitin sulfate, production-controlled and quality-controlled production of non-animal derived heparin has been required (NPL 1). As a specific example, a method for producing heparin having a structure and an anticoagulation activity equivalent to those of a pig-derived product by deacetylation and sulfation of heparosan, which is a capsular polysaccharide of a microorganism, through a chemical method and an enzymatic method has been reported (PTL land PTL 2).

In the natural process of biosynthesis of heparin in vivo, the modification that occurs after the formation of a heparosan-like disaccharide repeating sequence backbone is de-N-acetylation and N-sulfation of glucosamine, and these two reactions are carried out by the action of one polypeptide, N-deacetylase/N-sulfotransferase (hereinafter referred to as “NDST”) (NPL 1).

NDST is a family of 4 isozymes which have 65% to 80% identity to one another and have been cloned from various mammals (NPL 2). NDST is composed of an N-deacetylase domain and an N-sulfatase domain, and their activities vary depending on the origin and isozyme (NPL 2). The expression of NDST activity has been achieved in various eukaryotic systems such as COS cells, HEK 293 cells, CHO cells, etc., and also in insect cells and yeast (NPL 3 and NPL 4). On the other hand, although the expression of the N-sulfatase domain in Escherichia coli has been reported, the expression of active full-length NDST, especially the N-deacetylase domain, in bacteria has not been achieved (NPL 5). In recent years, a group at Jiangnan University has published literature and a patent on the expression of NDST activity in Pichia yeast, but it is also described therein that it is hard to confirm the deacetylation activity of NDST in Escherichia coli (PTL 3 and NPL 6).

### CITATION LIST

### Patent Literature

PTL 1: U.S. Pat. No. 8,771,995B
PTL 2: WO2018/048973
PTL 3: CN107384990B

### Non-Patent Literature

NPL 1: Advanced Drug Delivery Reviews 97 (2016) 237-249
NPL 2: Journal of Biological Chemistry 276 (2001) 5876-5882
NPL 3: Proceedings of the National Academy of Sciences of the United States of America 90 (1993) 3885-3888
NPL 4: Glycobiology 12 (2004) 1217-1228
NPL 5: FEBS Letters 433 (1998) 211-214
NPL 6: Green Chemistry 24 (2022) 3180-3192

### SUMMARY OF INVENTION

### Technical Problem

The N-deacetylation reaction of heparosan can also be chemically carried out, but if it can be carried out in a living organism, it can be carried out together with the subsequent O-sulfation reaction, which is considered to be more desirable in the production of non-animal-derived heparin (NPL 6). The expression of N-deacetylation reaction activity has already been achieved in Pichia yeast. On the other hand, the expression of N-deacetylation reaction activity has not been achieved in Escherichia coli, which has many achievements of expression of sulfotransferases, and the expression of the activity in bacteria including Escherichia coli has been desired.

In view of this, an object of the invention is to provide a novel method for producing a microorganism having N-deacetylation activity and N-sulfation activity, and the microorganism.

Heterologous expression of a gene can sometimes be improved by optimizing the nucleotide sequence of the gene according to the codon usage frequency of a host, and for example, in NPL 6, codon optimization is performed for the expression of various sulfotransferases. This utilizes the fact that the amount of tRNA differs even with the same amino acid codon depending on the host, and in consideration of this property, codon optimization should naturally be performed according to the codon usage frequency of the host, and it was generally believed that optimization according to the codon usage frequency of a living organism of another species would be suboptimal. For example, the codon usage frequency of budding yeast and the codon usage frequency of Escherichia coli are different, and in the case of a codon for arginine, CGU or CGC is preferred for Escherichia coli, but an AGA codon, which is rarely present in Escherichia coli is preferred for budding yeast. Therefore, it is unusual to attempt to perform codon optimization according to budding yeast when heterologous expression is performed using Escherichia coli as a host.

### Solution to Problem

The present inventors conducted intensive studies for achieving the above object, and as a result, they discovered that introducing NDST into a microorganism, using a sequence that had been subjected to codon optimization according to a different biological species, a microorganism having N-deacetylation activity and N-sulfation activity can be produced. Further, they found that by expressing a specific protein in a microorganism, a microorganism having N-deacetylation activity and N-sulfation activity can be produced.

That is, the present invention relates to the followings.

((1)) A method for producing a microorganism having N-deacetylation activity and N-sulfation activity, comprising:
a step (I) of modifying a nucleotide sequence of a DNA encoding N-deacetylase/N-sulfotransferase; and
a step (II) of introducing the DNA containing the nucleotide sequence modified in the step (I) into a microorganism in an expressible manner, wherein
the step (I) is a step of performing codon optimization according to the codon usage frequency of a biological species different from the microorganism.

((2)) The method for producing a microorganism having N-deacetylation activity and N-sulfation activity according to above ((1)), wherein the microorganism is a bacterium, and the biological species is budding yeast.

((3)) The method for producing a microorganism having N-deacetylation activity and N-sulfation activity according to above ((2)), wherein the DNA containing the modified nucleotide sequence is a DNA in (A-1) or (A-2):
(A-1) a DNA that contains the nucleotide sequence represented by SEQ ID NO: 6; or
(A-2) a DNA that contains a nucleotide sequence having 80% or more identity to the nucleotide sequence represented by SEQ ID NO: 6, and that encodes a protein having N-deacetylation activity and N-sulfation activity.

((4)) A method for producing a heparosan-derived compound, comprising a step (III) of producing an N-deacetylated and N-sulfated heparosan-derived compound from heparosan in the presence of a microorganism having N-deacetylation activity and N-sulfation activity produced by the production method according to above ((1)) or an extract thereof.

((5)) The production method according to above ((1)), wherein the microorganism is a bacterium of the genus Escherichia.

((6)) The production method according to above ((5)), wherein the bacterium of the genus Escherichia is Escherichia coli.

((7)) The production method according to above ((4)), wherein the heparosan-derived compound is N-sulfated heparosan.

((8)) A method for producing a microorganism having N-deacetylation activity and N-sulfation activity, comprising a step (i) of expressing any one protein selected from the group consisting of the following (B-1) to (B-3), (C-1) to (C-3), (D-1) to (D-3) and (E-1) to (E-3) in a microorganism:
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

((9)) The method for producing a microorganism having N-deacetylation activity and N-sulfation activity according to above ((8)), wherein
the step (i) is a step of introducing any one DNA selected from the group consisting of the following (b-1) to (b-3), (c-1) to (c-3), (d-1) to (d-3) and (e-1) to (e-3) into the microorganism in an expressible manner:
(b-1) a DNA that contains the nucleotide sequence represented by SEQ ID NO: 9;
(b-2) a DNA that encodes a protein containing the amino acid sequence represented by SEQ ID NO: 13;
(b-3) a DNA that contains a nucleotide sequence having 80% or more identity to the nucleotide sequence represented by SEQ ID NO: 9, and that encodes a protein having N-deacetylation activity and N-sulfation activity;
(c-1) a DNA that contains the nucleotide sequence represented by SEQ ID NO: 10;
(c-2) a DNA that encodes a protein containing the amino acid sequence represented by SEQ ID NO: 14;
(c-3) a DNA that contains a nucleotide sequence having 80% or more identity to the nucleotide sequence represented by SEQ ID NO: 10, and that encodes a protein having N-deacetylation activity and N-sulfation activity;
(d-1) a DNA that contains the nucleotide sequence represented by SEQ ID NO: 11;
(d-2) a DNA that encodes a protein containing the amino acid sequence represented by SEQ ID NO: 15;
(d-3) a DNA that contains a nucleotide sequence having 80% or more identity to the nucleotide sequence represented by SEQ ID NO: 11, and that encodes a protein having N-deacetylation activity and N-sulfation activity;
(e-1) a DNA that contains the nucleotide sequence represented by SEQ ID NO: 12;
(e-2) a DNA that encodes a protein containing the amino acid sequence represented by SEQ ID NO: 16; and
(e-3) a DNA that contains a nucleotide sequence having 80% or more identity to the nucleotide sequence represented by SEQ ID NO: 12, and that encodes a protein having N-deacetylation activity and N-sulfation activity.

((10)) A method for producing a heparosan-derived compound, comprising a step (ii) of producing an N-deacetylated and N-sulfated heparosan-derived compound from heparosan in the presence of a microorganism having N-deacetylation activity and N-sulfation activity produced by the production method according to above ((8)) or an extract thereof.

((11)) The production method according to above ((8)), wherein the microorganism is a bacterium of the genus Escherichia.

((12)) The production method according to above ((11)), wherein the bacterium of the genus Escherichia is Escherichia coli.

((13)) The production method according to above ((10)), wherein the heparosan-derived compound is N-sulfated heparosan.

((14)) A microorganism having N-deacetylation activity and N-sulfation activity, which expresses any one protein selected from the group consisting of the following (B-1) to (B-3), (C-1) to (C-3), (D-1) to (D-3) and (E-1) to (E-3):
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

### Advantageous Effects of Invention

In a production method according to a first aspect of the invention, by introducing NDST having been subjected to codon optimization according to a different biological species into a microorganism, a microorganism having excellent N-deacetylation activity and N-sulfation activity as compared to the related art can be produced.

In a production method according to a second aspect of the invention, by expressing a protein having a specific amino acid sequence in a microorganism, a microorganism having excellent N-deacetylation activity and N-sulfation activity as compared to the related art can be produced.

### BRIEF DESCRIPTION OF DRAWINGS

FIG. 1 shows the results of a constituent disaccharide amount analysis in an NDST reaction test under the condition that heparosan was used as a substrate and PAPS was not added.

FIG. 2 shows the results of a constituent disaccharide amount analysis in an NDST reaction test under the condition that deacetylated heparosan was used as a substrate and PAPS was added.

FIG. 3 shows the results of a constituent disaccharide amount analysis in an NDST reaction test under the condition that heparosan was used as a substrate and PAPS was added.

### DESCRIPTION OF EMBODIMENTS

Hereinafter, the invention will be described in detail, but these show examples of preferred embodiments and the invention is not limited to these contents.

The numerical range indicated by using “to” is a range including numerical values before and after “to”, and for example, “0 mass % to 100 mass %” means a range of 0 mass % or more and 100 mass % or less.

### 1. Method for Producing Microorganism Having N-Deacetylation Activity and N-Sulfation Activity (1)

The method for producing a microorganism having N-deacetylation activity and N-sulfation activity (hereinafter also referred to as “microorganism expressing NDST activity”) according to one embodiment of the invention includes:
a step (I) of modifying a nucleotide sequence of a DNA encoding NDST; and
a step (II) of introducing the DNA containing the nucleotide sequence modified in the step (I) into a microorganism in an expressible manner, wherein
the step (I) is a step of performing codon optimization according to the codon usage frequency of a biological species different from the microorganism.

In the present specification, the “N-deacetylation” means, for example, that an amino group is produced by N-deacetylation of an N-acetyl group of an alpha-D-glucosamine residue of heparosan. Examples of the N-deacetylation include partial N-deacetylation.

In addition, in the present specification, the “N-sulfation” means, for example, that an amino group of an N-acetyl-D-glucosamine residue of heparosan is sulfated.

Examples of the nucleotide sequence of a DNA encoding NDST used in the step (I) include the nucleotide sequence represented by SEQ ID NO: 1.

In the step (I) of modifying the nucleotide sequence of a DNA encoding NDST, codon optimization is performed according to the codon usage frequency of a biological species different from the microorganism (hereinafter also referred to as “parent strain”) into which the DNA is introduced in the step (II). In general, the “codon optimization” refers to changing of a codon for each amino acid forming a peptide to a codon with a high usage frequency in the biological species into which the DNA is introduced. In the invention, it was surprisingly found that by performing codon optimization according to the codon usage frequency of a biological species different from the microorganism into which the DNA is introduced, the microorganism expressing NDST activity can be obtained. This result implies that this species mismatched codon optimization brings active expression of NDST by slowing the protein expression and avoiding aggregation in the cells.

Here, the “codon optimization” does not necessarily refer to changing of the codons for 100% of the amino acids in the amino acid sequence of a polypeptide, but refers to changing of a codon corresponding to at least one amino acid. However, it is preferred that 50% or more codons are changed.

In the above step (I), at least one codon among the codons corresponding to the nucleotide sequence of a DNA encoding NDST is preferably subjected to codon optimization according to the codon usage frequency of a biological species different from the parent strain.

The biological species different from the microorganism (parent strain) into which the DNA is introduced in the above step (II) is preferably yeast, an insect cell, and a filamentous fungus, and particularly preferably budding yeast from the viewpoint of past achievements of heterologous expression. The codon with a high usage frequency in these biological species can be confirmed by, for example, Codon Usage Database (https://www.kazusa.or.jp/codon/). A codon optimization method for each biological species is well known to those skilled in the art.

In the present specification, the parent strain refers to the original strain to be subjected to genetic modification, transformation, or the like. In particular, the parent strain of the microorganism having N-deacetylation activity and N-sulfation activity of the invention in the “Method for producing microorganism having N-deacetylation activity and N-sulfation activity (1)” refers to a strain into which the DNA containing the modified nucleotide sequence is introduced in an expressible manner in the above step (II).

From the viewpoint of ease of industrial culturing, the species of the microorganism (parent strain) into which the DNA is introduced in an expressible manner in the above step (II) is preferably a bacterium belonging to the genus Escherichia, the genus Serratia, the genus Bacillus, the genus Brevibacterium, the genus Corynebacterium. the genus Microbacterium, the genus Pseudomonas, or the like, more preferably a bacterium of the genus Escherichia, and particularly preferably Escherichia coli.

Specific non-limiting examples of the Escherichia coli include Escherichia coli Origami B (DE3) (manufactured by Novagen), Escherichia coli BL21 codon plus, Escherichia coli XL1-Blue, Escherichia coli XL2-Blue (all manufactured by Agilent Technologies, Inc.), Escherichia coli BL21 (DE3)pLysS (manufactured by Merck Millipore), Escherichia coli BL21, Escherichia coli DH5 alpha, Escherichia coli HST08 Premium, Escherichia coli HST02, Escherichia coli HST04 dam-/dcm-, Escherichia coli JM109, Escherichia coli HB101, Escherichia coli CJ236, Escherichia coli BMH 71-18 mutS, Escherichia coli MV1184, Escherichia coli TH2 (all manufactured by Takara Bio Inc.), Escherichia coli W (ATCC9637), Escherichia coli B (ATCC23226), Escherichia coli JM101, Escherichia coli W3110, Escherichia coli MG 1655, Escherichia coli DH1, Escherichia coli MC1000, Escherichia coli W1485, Escherichia coli MP347, Escherichia coli NM522, Escherichia coli K5, and Escherichia coli Nissle 1917.

The parent strain used in the invention may be one that inherently has a heparosan-producing ability, or one that has been modified to have a heparosan-producing ability. A microorganism having a heparosan-producing ability can be obtained by imparting a heparosan-producing ability to a microorganism as described above.

The heparosan-producing ability can be imparted by introducing a gene encoding a protein involved in heparosan production with reference to Metabolic Engineering, 2012, 14, pp. 521-527, Carbohydrate Research, 2012, 360, pp. 19-24, U.S. Pat. No. 9,975,928B, or the like. Examples of the protein involved in heparosan production include a glycosyltransferase and a heparosan efflux carrier protein. In the parent strain used in the invention, one type of gene may be introduced, or two or more types of genes may be introduced. The introduction of a gene can also be achieved by introducing a vector containing the gene into a host. For example, a DNA fragment containing a target gene is ligated to a vector that functions in a host to construct an expression vector for the gene, and the host is transformed with the expression vector, whereby the copy number of the gene can be increased. The DNA fragment containing the target gene can be obtained, for example, by PCR using a genome DNA of a microorganism having the target gene as a template. The transformation method is not particularly limited, and a conventionally known method can be used.

The introduction of the DNA containing the nucleotide sequence modified in the step (I) into a microorganism in an expressible manner in the step (II) is preferably transformation of the microorganism (parent strain) with a recombinant DNA having the DNA containing the nucleotide sequence modified in the step (I).

The DNA containing the nucleotide sequence modified in the above step (I) is preferably a DNA in the following (A-1) or (A-2).
(A-1) a DNA that contains the nucleotide sequence represented by SEQ ID NO: 6
(A-2) a DNA that contains a nucleotide sequence having 80% or more identity to the nucleotide sequence represented by SEQ ID NO: 6, and that encodes a protein having N-deacetylation activity and N-sulfation activity (hereinafter also referred to as “NDST activity”)

When the DNA containing the nucleotide sequence modified in the above step (I) is the DNA in the above (A-1) or (A-2), the biological species different from the parent strain in the above step (II) is budding yeast.

Examples of the DNA described in the above (A-2) include a DNA that is composed of a nucleotide sequence having 80% or more, preferably in the following order of 85% or more, 90% or more, 95% or more, more preferably 98% or more, and most preferably 99% or more identity to the nucleotide sequence represented by SEQ ID NO: 6, and that encodes a protein having NDST activity.

In the present specification, the identity of nucleotide sequences or amino acid sequences can be determined using the Lipman-Pearson method [Science, 227 (4693), 1435-41 (1985)], algorithm BLAST by Karlin and Altschul [Pro. Natl. Acad. Sci. USA, 90, 5873 (1993)] or FASTA [Methods Enzymol., 183, 63 (1990)]. Based on the algorithm BLAST, a program called BLASTN or BLASTX has been developed [J. Mol. Biol., 215, 403 (1990)]. When a nucleotide sequence is analyzed by BLASTN based on BLAST, the parameters are set, for example, as follows: score=100 and wordlength=12. In addition, when an amino acid sequence is analyzed by BLASTX based on BLAST, the parameters are set, for example, as follows: score=50 and wordlength=3. When BLAST and Gapped BLAST programs are used, default parameters of each program are used. A specific method of such an analytical method is known.

The DNA described in the above (A-1) can be prepared, for example, by chemical synthesis based on the nucleotide sequence represented by SEQ ID NO: 6 using an NTS M series DNA synthesizer manufactured by Nihon Techno Service Co., Ltd. or the like.

The DNA described in the above (A-2) can be prepared, for example, by searching for a nucleotide sequence having 80% or more, preferably in the following order of 85% or more, 90% or more, 95% or more, preferably 97% or more, more preferably 98% or more, and most preferably 99% or more identity to the nucleotide sequence represented by SEQ ID NO: 6 in various gene sequence databases, and performing chemical synthesis based on the nucleotide sequence obtained by the search using an NTS M series DNA synthesizer manufactured by Nihon Techno Service Co., Ltd. or the like.

The recombinant DNA having the DNA containing the nucleotide sequence modified in the above step (I), preferably the DNA described in the above (A-1) or (A-2) refers to, for example, a DNA, in which the DNA is autonomously replicable in the parent strain, and the DNA containing the nucleotide sequence modified in the above step (I), preferably the DNA in which the DNA containing the nucleotide sequence modified in the above step (I), preferably the DNA described in the above (A-1) or (A-2) is integrated into an expression vector containing a promoter at a position where the DNA described in the above (A-1) or (A-2) can be transcribed.

The DNA which can be integrated into the chromosome of the parent strain and is the DNA containing the nucleotide sequence modified in the above step (I), preferably the DNA described in the above (A-1) or (A-2) is also the recombinant DNA having the DNA containing the nucleotide sequence modified in the above step (I), preferably the DNA described in the above (A-1) or (A-2). The recombinant DNA need not contain a promoter when it is a DNA which can be integrated into the chromosomal DNA of the parent strain.

When a prokaryote such as a bacterium is used as the parent strain, the recombinant DNA which is autonomously replicable in the parent strain is preferably a recombinant DNA composed of a promoter, a ribosomal binding sequence, the nucleotide sequence modified in the above step (I), preferably the DNA described in the above (A-1) or (A-2), and a transcription termination sequence. It may contain a gene that controls the promoter. It is preferred to use a recombinant DNA in which a distance between a Shine-Dalgarno sequence that is a ribosomal binding sequence and a start codon is appropriately adjusted (for example, 6 to 18 nucleotides).

In the recombinant DNA which is autonomously replicable in the parent strain, the transcription termination sequence is not necessarily needed for the expression of the DNA, but the transcription termination sequence is preferably placed immediately downstream of a structural gene.

In the present specification, the expression vector is not particularly limited as long as it is a suitable nucleic acid molecule for introducing, proliferating, and expressing a target DNA in a host, and not only a plasmid but also a vector using an artificial chromosome or a transposon, or a cosmid may be used.

In the present specification, when a microorganism belonging to the genus Escherichia is used as the parent strain, examples of the expression vector include pColdI, pSTV28, pSTV29, pUC118 (all manufactured by Takara Bio Inc.), pMW119 (manufactured by Nippon Gene Co., Ltd.), pET21a, pCOLADuet-1, pCDFDuet-1, pCDF-1b, pRSF-1b (all manufactured by Merck Millipore), pMAL-c5x (manufactured by New England Biolabs, Inc.), pGEX-4T-1, pTrc99A (both manufactured by GE Healthcare Bio-Sciences Corporation), pTrcHis, pSE280 (both manufactured by Thermo Fisher Scientific, Inc.), pGEMEX-1 (manufactured by Promega Corporation), pQE-30, pQE80L (both manufactured by QIAGEN N.V.), pET-3, pBluescriptII SK (+), pBluescriptII KS (−) (all manufactured by Agilent Technologies, Inc.), pKYP10 (JPS58-110600A), pKYP200 [Agric. Biol. Chem., 48, 669 (1984)], pLSA1 [Agric. Biol. Chem., 53, 277 (1989)], pGEL1 [Proc. Natl. Acad. Sci., USA, 82, 4306 (1985)], pTrS30 [prepared from Escherichia coli JM109/pTrS30 (FERM BP-5407)], pTrS32 [prepared from Escherichia coli JM109/pTrS32 (FERM BP-5408)], pTK31 [Applied and Environmental Microbiology, 2007, Vol. 73, No. 20, pp. 6378-6385], pPAC31 (WO98/12343), pUC19 [Gene, 33, 103 (1985)], and pPA1 (JPS63-233798A).

As the promoter when using the above-mentioned expression vector, any promoter may be used as long as it functions in a cell of a microorganism belonging to the genus Escherichia, and examples thereof include promoters for genes involved in amino acid biosynthesis such as a trp promoter and an ilv promoter, and promoters derived from Escherichia coli, a phage, or the like such as a uspA promoter, a lac promoter, a PL promoter, a PR promoter, and a PSE promoter. In addition, a promoter that is artificially designed and modified such as a promoter in which two trp promoters are linked in tandem, a tac promoter, a trc promoter, a lacT7 promoter, or a letI promoter can also be used.

The recombinant DNA used in the invention described in the present specification can be prepared, for example, by treating a DNA fragment prepared by the abovementioned method with a restriction enzyme or the like, and inserting it downstream of the promoter of the above-mentioned appropriate expression vector.

In the present specification, as a method for introducing a recombinant DNA having a DNA encoding a protein having N-deacetylation activity and N-sulfation activity into a plasmid which is autonomously replicable in a host cell, for example, a method using calcium ions, a protoplast method, an electroporation method, a spheroplast method [Proc. Natl. Acad. Sci., USA, 81, 4889 (1984)], a lithium acetate method [J. Bacteriol., 153, 163 (1983)], and the like are exemplified.

Further, in the present specification, when a recombinant DNA having a DNA encoding a protein having N-deacetylation activity and N-sulfation activity is inserted into the genome of a parent strain, for example, a method using homologous recombination may be used. That is, a DNA to which a part of a chromosomal region that causes introduction of a target DNA is attached is incorporated in a microbial cell to cause homologous recombination in the partial region of the chromosomal region, whereby the DNA can be integrated into the genome. For example, as a method using homologous recombination frequently used in Escherichia coli, a method of introducing a recombinant DNA using a homologous recombination system of a lambda phage [Proc. Natl. Acad. Sci. USA, 97, 6641-6645 (2000)] is exemplified. Here, the chromosomal region that causes the introduction is not particularly limited, but is preferably a non-essential gene region or a non-gene region upstream of a nonessential gene region. As a method for incorporating the DNA in a microbial cell, any method can be used as long as it is a method for introducing a DNA into a host cell, and examples thereof include a method using calcium ions, a protoplast method, and an electroporation method described above.

In the present specification, whether it is a microorganism obtained by introducing a recombinant DNA having a DNA encoding a protein having N-deacetylation activity and N-sulfation activity into the parent strain in an expressible manner can be confirmed, for example, by comparing the transcription amount of the DNA in the microorganism by Northern blotting or the production amount of the protein in the microorganism by Western blotting with that in the parent strain.

In the present specification, whether a microorganism produced by the abovementioned method is a recombinant microorganism having N-deacetylation activity and N-sulfation activity can be confirmed, for example, by the following method. First, each of the parent strain and the produced microorganism is cultured in a medium, and a cell extract solution containing a protein having N-deacetylation activity and N-sulfation activity is prepared from the obtained culture. Subsequently, the cell extract solution is brought into contact with heparosan which is a substrate and PAPS which is a sulfate group donor to produce N-deacetylated and N-sulfated heparosan. Finally, by detecting N-deacetylated and N-sulfated heparosan in the reaction solution through a general analytical method using high-performance liquid chromatography, gas chromatography, or the like, it can be confirmed that the produced microorganism is a recombinant microorganism having N-deacetylation activity and N-sulfation activity.

### 2. Method for Producing Microorganism Having N-Deacetylation Activity and N-Sulfation Activity (2)

The method for producing a microorganism expressing NDST activity according to one embodiment of the invention includes a step (i) of expressing any one protein selected from the group consisting of the following (B-1) to (B-3), (C-1) to (C-3), (D-1) to (D-3) and (E-1) to (E-3) in a microorganism:
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has NDST activity;
(B-3) a protein that contains an amino acid sequence in which one or several amino acid residues have been deleted, substituted, added, or inserted in the amino acid sequence represented by SEQ ID NO: 13, and that has NDST activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has NDST activity;
(C-3) a protein that contains an amino acid sequence in which one or several amino acid residues have been deleted, substituted, added, or inserted in the amino acid sequence represented by SEQ ID NO: 14, and that has NDST activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has NDST activity; and
(E-3) a protein that contains an amino acid sequence in which one or several amino acid residues have been deleted, substituted, added, or inserted in the amino acid sequence represented by SEQ ID NO: 16, and that has NDST activity.

As the protein described in the above (B-2), (C-2), (D-2), or (E-2), a protein that is composed of an amino acid sequence having 80% or more, preferably in the following order of 85% or more, 90% or more, 95% or more, more preferably 98% or more, and most preferably 99% or more identity to the amino acid sequence represented by SEQ ID NO: 13, 14, 15, or 16, respectively, and that has NDST activity is exemplified.

As the protein described in the above (B-3), (C-3), (D-3), or (E-3), a protein that contains an amino acid sequence in which one or several, preferably 1 to 20, more preferably 1 to 10, even more preferably 1 to 8, and most preferably 1 to 5 amino acids have been deleted, substituted, added, or inserted in the amino acid sequence represented by SEQ ID NO: 13, 14, 15, or 16, respectively, and that has NDST activity is exemplified.

In order to obtain a microorganism having high N-deacetylation activity and N-sulfation activity, in the above step (i), it is preferred to express any one protein selected from the group consisting of (B-1) to (B-3), (C-1) to (C-3) and (E-1) to (E-3) among the above-mentioned proteins in a microorganism, and it is more preferred to express any one protein selected from the group consisting of (C-1) to (C-3) among the above-mentioned proteins in a microorganism.

The amino acid sequence in which an amino acid has been deleted, substituted, added, or inserted refers to an amino acid sequence obtained by artificially deleting or substituting an amino acid residue in the original amino acid sequence or artificially adding or inserting an amino acid residue in the amino acid sequence.

It does not matter whether the amino acid to be deleted, substituted, added, or inserted is a natural type or an unnatural type. Examples of the natural type amino acid include L-alanine, L-asparagine, L-aspartic acid, L-glutamine, L-glutamic acid, glycine, L-histidine, L-isoleucine, L-leucine, L-lysine, L-arginine, L-methionine, L-phenylalanine, L-proline, L-serine, L-threonine, L-tryptophan, L-tyrosine, L-valine, and L-cysteine.

Hereinafter, examples of mutually substitutable amino acids are shown. Amino acids included in the same group can be mutually substituted.
group A: leucine, isoleucine, norleucine, valine, norvaline, alanine, 2-aminobutanoic acid, methionine, O-methylserine, t-butyl glycine, t-butyl alanine, and cyclohexylalanine
group B: aspartic acid, glutamic acid, isoaspartic acid, isoglutamic acid, 2-aminoadipic acid, and 2-aminosuberic acid
group C: asparagine and glutamine
group D: lysine, arginine, ornithine, 2,4-diaminobutanoic acid, and 2,3-diaminopropionic acid
group E: proline, 3-hydroxyproline, and 4-hydroxyproline
group F: serine, threonine, and homoserine
group G: phenylalanine, tryptophan, and tyrosine

Whether the protein has N-deacetylation activity and N-sulfation activity (NDST activity) can be confirmed, for example, by the following method.

First, a recombinant DNA containing a DNA encoding the protein is prepared by the below-mentioned method. Subsequently, a microorganism obtained by transforming a microorganism in which the NDST activity cannot be confirmed, for example, Escherichia coli Origami B (DE3) with the recombinant DNA is cultured, and a cell extract solution containing the protein is prepared from the obtained culture. Subsequently, the cell extract solution is allowed to react in the presence of 3′-phosphoadenosine-5′-phosphosulfate (hereinafter also referred to as “PAPS”) using heparosan as a substrate, and finally, a general analytical method using high-performance liquid chromatography, gas chromatography, or the like is performed. By detecting the production of N-deacetylated and N-sulfated heparosan, it can be confirmed that the protein of interest has N-deacetylation activity and N-sulfation activity.

A preferred microbial species as the microorganism (hereinafter also referred to as “parent strain”) used in the above step (i) is the same as the preferred microbial species exemplified as the microorganism (parent strain) into which a DNA is introduced in an expressible manner in the step (II) in the above 1.

In the present specification, the parent strain refers to the original strain to be subjected to genetic modification, transformation, or the like. In particular, the parent strain of the microorganism having N-deacetylation activity and N-sulfation activity of the invention in “Method for producing microorganism having N-deacetylation activity and N-sulfation activity (2)” refers to a strain before expressing any one protein selected from the group consisting of (B-1) to (B-3), (C-1) to (C-3), (D-1) to (D-3) and (E-1) to (E-3) in the above step (i).

The above step (i) is preferably a step of introducing any one DNA selected from the group consisting of the following (b-1) to (b-3), (c-1) to (c-3), (d-1) to (d-3) and (e-1) to (e-3) into the microorganism in an expressible manner:
(b-1) a DNA that contains the nucleotide sequence represented by SEQ ID NO: 9;
(b-2) a DNA that encodes a protein containing the amino acid sequence represented by SEQ ID NO: 13;
(b-3) a DNA that contains a nucleotide sequence having 80% or more identity to the nucleotide sequence represented by SEQ ID NO: 9, and that encodes a protein having N-deacetylation activity and N-sulfation activity;
(c-1) a DNA that contains the nucleotide sequence represented by SEQ ID NO: 10;
(c-2) a DNA that encodes a protein containing the amino acid sequence represented by SEQ ID NO: 14;
(c-3) a DNA that contains a nucleotide sequence having 80% or more identity to the nucleotide sequence represented by SEQ ID NO: 10, and that encodes a protein having N-deacetylation activity and N-sulfation activity;
(d-1) a DNA that contains the nucleotide sequence represented by SEQ ID NO: 11;
(d-2) a DNA that encodes a protein containing the amino acid sequence represented by SEQ ID NO: 15;
(d-3) a DNA that contains a nucleotide sequence having 80% or more identity to the nucleotide sequence represented by SEQ ID NO: 11, and that encodes a protein having N-deacetylation activity and N-sulfation activity;
(e-1) a DNA that contains the nucleotide sequence represented by SEQ ID NO: 12;
(e-2) a DNA that encodes a protein containing the amino acid sequence represented by SEQ ID NO: 16; and (e-3) a DNA that contains a nucleotide sequence having 80% or more identity to the nucleotide sequence represented by SEQ ID NO: 12, and that encodes a protein having N-deacetylation activity and N-sulfation activity.

In order to obtain a microorganism having high N-deacetylation activity and N-sulfation activity, in the above step (i), it is preferred to introduce any one DNA selected from the group consisting of (b-1) to (b-3), (c-1) to (c-3) and (e-1) to (e-3) among the above-mentioned DNAs into a microorganism in an expressible manner, and it is more preferred to introduce any one DNA selected from the group consisting of (c-1) to (c-3) among the above-mentioned DNAs into a microorganism in an expressible manner.

The DNAs described in the above (b-1), (c-1), (d-1), (e-1), (b-2), (c-2), (d-2), and (e-2) can be prepared, for example, by chemical synthesis based on the nucleotide sequence represented by SEQ ID NO: 9, 10, 11, or 12 and the amino acid sequence represented by SEQ ID NO: 13, 14, 15, or 16, respectively, using an NTS M series DNA synthesizer manufactured by Nihon Techno Service Co., Ltd. or the like.

The DNA described in the above (b-3), (c-3), (d-3), or (e-3) can be prepared, for example, by searching for a nucleotide sequence having 80% or more, preferably in the following order of 87% or more, 88% or more, 90% or more, 95% or more, more preferably 98% or more, and most preferably 99% or more identity to the nucleotide sequence represented by SEQ ID NO: 9, 10, 11, or 12, respectively, in various gene sequence databases, and performing chemical synthesis based on the nucleotide sequence obtained by the search using an NTS M series DNA synthesizer manufactured by Nihon Techno Service Co., Ltd. or the like.

Examples of the introduction of any one DNA selected from the group consisting of the above (b-1) to (b-3), (c-1) to (c-3), (d-1) to (d-3) and (e-1) to (e-3) into a microorganism in an expressible manner in the above step (i) include transformation of the microorganism (parent strain) with a recombinant DNA having any one DNA selected from the group consisting of the above (b-1) to (b-3), (c-1) to (c-3), (d-1) to (d-3) and (e-1) to (e-3).

The recombinant DNA having a DNA described in any one of the above (b-1) to (b-3), (c-1) to (c-3), (d-1) to (d-3) and (e-1) to (e-3) refers to, for example, a DNA, in which the DNA is autonomously replicable in the parent strain, and the DNA described in any one or more of the above (b-1) to (b-3), (c-1) to (c-3), (d-1) to (d-3) and (e-1) to (e-3) is integrated into an expression vector containing a promoter at a position where the DNA described in any one or more of the above (b-1) to (b-3), (c-1) to (c-3), (d-1) to (d-3) and (e-1) to (e-3) can be transcribed.

The DNA which can be integrated into the chromosome of the parent strain and is the DNA described in any one or more of the above (b-1) to (b-3), (c-1) to (c-3), (d-1) to (d-3) and (e-1) to (e-3) is also the recombinant DNA having the DNA described in any one or more of the above (b-1) to (b-3), (c-1) to (c-3), (d-1) to (d-3) and (e-1) to (e-3). The recombinant DNA need not contain a promoter when it is a DNA which can be integrated into the chromosomal DNA of the parent strain.

When a prokaryote such as a bacterium is used as the parent strain, the recombinant DNA which is autonomously replicable in the parent strain is preferably a recombinant DNA composed of a promoter, a ribosomal binding sequence, the DNA described in any one or more of the above (b-1) to (b-3), (c-1) to (c-3), (d-1) to (d-3) and (e-1) to (e-3), and a transcription termination sequence. It may contain a gene that controls the promoter. It is preferred to use a recombinant DNA in which a distance between a Shine-Dalgarno sequence that is a ribosomal binding sequence and a start codon is appropriately adjusted (for example, 6 to 18 nucleotides).

In the recombinant DNA which is autonomously replicable in the parent strain, the transcription termination sequence is not necessarily needed for the expression of the DNA, but the transcription termination sequence is preferably placed immediately downstream of a structural gene.

### 3. Microorganism Having N-Deacetylation Activity and N-Sulfation Activity

The microorganism having N-deacetylation activity and N-sulfation activity according to one embodiment of the invention expresses any one protein selected from the group consisting of the following (B-1) to (B-3), (C-1) to (C-3), (D-1) to (D-3) and (E-1) to (E-3):
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14; (C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15; (D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16; (E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

The microorganism having N-deacetylation activity and N-sulfation activity described above can be produced by the method in the above 2. A preferred microbial species as the microorganism is the same as the preferred microbial species as the microorganism used in the step (i) in the above 2.

### 4. Method for Producing Heparosan-Derived Compound

One embodiment of the method for producing a heparosan-derived compound of the invention includes a step (III) of producing an N-deacetylated and N-sulfated heparosan-derived compound from heparosan in the presence of a microorganism having N-deacetylation activity and N-sulfation activity produced by the above steps (I) and (II) in the above 1 or an extract thereof.

Further, one embodiment of the method for producing a heparosan-derived compound of the invention includes a step (ii) of producing an N-deacetylated and N-sulfated heparosan-derived compound from heparosan in the presence of a microorganism having N-deacetylation activity and N-sulfation activity produced by the above step (i) in the above 2 or an extract thereof.

The heparosan-derived compound to be produced is preferably N-sulfated heparosan, N-sulfated and epimerized heparosan, N-sulfated and depolymerized heparosan, N-sulfated, epimerized, and depolymerized heparosan, N-sulfated-6-O-sulfated heparosan, N-sulfated-6-O-sulfated and epimerized heparosan, N-sulfated-2-O-sulfated-6-O-sulfated heparosan, N-sulfated-2-O-sulfated-6-O-sulfated, epimerized, and depolymerized heparosan, N-sulfated-6-O-sulfated, and depolymerized heparosan, N-sulfated-6-O-sulfated, epimerized, and depolymerized heparosan, N-sulfated-2-O-sulfated-6-O-sulfated and depolymerized heparosan, N-sulfated-2-O-sulfated-6-O-sulfated, epimerized, and depolymerized heparosan or heparin, and more preferably N-sulfated heparosan or heparin, and most preferably N-sulfated heparosan.

The “epimerization” means that a beta-D-glucuronic acid residue of heparosan is converted to an alpha-L-iduronic acid residue.

The “depolymerization” means that a treatment is performed so as to decrease the molecular weight. For example, the “depolymerized” heparosan compound has a number average molecular weight (Mn) of 1,000 to 150,000, preferably 8,000 to 60,000, and a weight average molecular weight (Mw) of 2,000 to 300,000, preferably 10,000 to 100,000 as a value measured by GPC using pullulan as a standard.

The “6-O-sulfation” means that a hydroxy group at the 6-position of an N-acetyl-D-glucosamine residue is sulfated.

The “2-O-sulfation” means that a hydroxy group at the 2-position of a hexuronic acid residue (preferably an alpha-L-iduronic acid residue) is sulfated.

Examples of the method for producing a heparosan-derived compound of the invention include (alpha) a method for producing an N-deacetylated and N-sulfated heparosan-derived compound by a fermentation method, and (beta) a method for producing an N-deacetylated and N-sulfated heparosan-derived compound by adding a culture of a microorganism having N-deacetylation activity and N-sulfation activity or a treated material of the culture to heparosan which is a substrate to cause a reaction. Hereinafter, the respective production methods will be described.

### (Alpha) Method for Producing N-Deacetylated and N-Sulfated Heparosan-Derived Compound by Fermentation Method

The production of an N-deacetylated and N-sulfated heparosan-derived compound by a fermentation method can be carried out by a method in which a microorganism having N-deacetylation activity and N-sulfation activity is cultured in a medium and allowed to produce the N-deacetylated and N-sulfated heparosan-derived compound in a culture. The production method may include, for example, producing and then accumulating the N-deacetylated and N-sulfated heparosan-derived compound in a culture and collecting the N-deacetylated and N-sulfated heparosan-derived compound from the culture.

The recombinant microorganism used in the method for producing an N-deacetylated and N-sulfated heparosan-derived compound by a fermentation method is a microorganism produced by the above steps (I) and (II) or a microorganism produced by the above step (i), and also is preferably a microorganism having an ability to produce heparosan to serve as a substrate for a protein having NDST activity, and/or a microorganism having an artificially enhanced ability to produce PAPS which is a sulfate group donor. The microorganism may be a microorganism having one or more activities of epimerization, 2-O-sulfation, 6-O-sulfation, and 3-O-sulfation.

The heparosan-producing ability can be imparted by introducing a gene encoding a protein involved in heparosan production by referring to Metabolic Engineering 2012, 14, pp. 521-527, Carbohydrate Research 2012, 360, pp. 19-24, U.S. Pat. No. 9,975,928B, or the like. The ability to produce PAPS can be imparted by introducing a gene encoding a protein involved in PAPS production by referring to J. Org. Chem. 2000, 65, 18, 5565-5574, WO2021/201282, or the like. The activity of epimerization, 2-O-sulfation, 6-O-sulfation, or 3-O-sulfation can be imparted by introducing a gene encoding C5-epimerase, 2-O-sulfotransferase, 6-O-sulfotransferase, 3-O-sulfotransferase, including relevant isoforms, or the like by referring to WO2021/201282 or the like.

The culturing of the microorganism can be carried out according to a usual method. As the medium for culturing the microorganism, either a natural medium or a synthetic medium may be used as long as it is a medium, which contains heparosan and a carbon source, a nitrogen source, an inorganic salt, and the like that can be assimilated by the microorganism, and with which the culturing of the microorganism can be efficiently performed.

The carbon source may be any as long as it can be assimilated by the microorganism, and examples thereof include glucose, fructose, sucrose, molasses containing these, carbohydrates such as starch and a starch hydrolysate, organic acids such as acetic acid and propionic acid, and alcohols such as ethanol, propanol, and glycerol.

Examples of the nitrogen source include ammonia, ammonium salts of inorganic acids or organic acids such as ammonium chloride, ammonium sulfate, ammonium acetate, and ammonium phosphate, other nitrogen-containing compounds, peptone, meat extract, yeast extract, corn steep liquor, a casein hydrolysate, soybean meal and a soybean meal hydrolysate, various fermentative bacterial cells, and digestive products thereof.

Examples of the inorganic salt include monopotassium phosphate, dipotassium phosphate, magnesium phosphate, magnesium sulfate, sodium chloride, ferrous sulfate, manganese sulfate, copper sulfate, and calcium carbonate.

In the method for producing a heparosan-derived compound by a fermentation method, when the microorganism to be used does not have an ability to produce heparosan to serve as a substrate for a protein having NDST activity, heparosan may be added to the medium.

Further, in the method for producing a heparosan-derived compound by a fermentation method, when the microorganism to be used does not have an ability to produce heparosan to serve as a substrate for a protein having NDST activity, heparosan may be supplied to the microorganism to be used in the invention by coculturing a microorganism having an ability to produce heparosan with the recombinant microorganism of the invention instead of adding heparosan to the medium.

In the method for producing a heparosan-derived compound by a fermentation method, when the microorganism to be used does not have an ability to supply PAPS necessary for the N-sulfation reaction, PAPS may be added to the medium.

Further, in the method for producing a heparosan-derived compound by a fermentation method, when the microorganism to be used does not have an ability to supply PAPS necessary for the N-sulfation reaction, PAPS may be supplied to the microorganism to be used in the invention by co-culturing a microorganism having an ability to produce PAPS with the recombinant microorganism of the invention instead of adding PAPS to the medium.

It is preferred that the culturing is usually performed under aerobic conditions such as shaking culture or deep aeration stirring culture. The culturing temperature is preferably 15 to 40 degree Celsius, and the culturing time is usually 5 hours to 7 days. The pH during culturing is preferably maintained at 3.0 to 9.0. The adjustment of the pH is performed using an inorganic or organic acid, an alkaline solution, urea, calcium carbonate, ammonia, or the like.

In addition, an antibiotic such as ampicillin or tetracycline may be added to the medium as needed during culturing. When culturing a microorganism transformed with an expression vector using an inducible promoter as a promoter, an inducer may be added to the medium as needed.

For example, isopropyl-beta-D-thiogalactopyranoside or the like may be added to the medium when culturing a microorganism transformed with an expression vector using a lac promoter, and indoleacrylic acid or the like may be added to the medium when culturing a microorganism transformed with an expression vector using a trp promoter.

A heparosan-derived compound can be produced by producing the heparosan-derived compound in a culture by the above-mentioned culturing. Quantification of the heparosan-derived compound can be performed using an HPLC (for example, an analyzer SPD-M20A manufactured by Shimadzu Corporation).

The collection of the heparosan-derived compound from the culture can usually be carried out by combining an ion-exchange resin method, a precipitation method, and other known methods. When the heparosan-derived compound is accumulated in bacterial cells, for example, the bacterial cells are disrupted by ultrasonic waves or the like, followed by centrifugation to remove the bacterial cells, and from the obtained supernatant, the heparosan-derived compound can be collected by an ion-exchange resin method or the like.

(beta) Method for producing N-deacetylated and N-sulfated heparosan-derived compound by adding culture of microorganism having N-deacetylation activity and N-sulfation activity or treated material of the culture to heparosan which is substrate to cause reaction

The production of the heparosan-derived compound described above can be carried out by a method in which a microorganism having N-deacetylation activity and N-sulfation activity or an extract thereof as an enzyme source, heparosan which is a substrate, and PAPS which is a sulfate group donor are made present in an aqueous medium, and an N-deacetylated and N-sulfated heparosan-derived compound is produced in the aqueous medium. The production method may include, for example, producing and then accumulating the N-deacetylated and N-sulfated heparosan-derived compound in an aqueous medium and collecting the N-deacetylated and N-sulfated heparosan-derived compound from the aqueous medium.

The method and the medium for culturing the microorganism are the same as those described above in (alpha).

In the present specification, examples of the treated material of the culture include a concentrate of the above culture, a dried material of the culture, bacterial cells obtained by subjecting the culture to centrifugation, filtration, or the like, a dried material of the bacterial cells, a lyophilizate of the bacterial cells, a surfactant-treated material of the bacterial cells, a solvent-treated material of the bacterial cells, an enzyme-treated material of the bacterial cells, a material containing viable bacterial cells that retain the same function as the culture as the enzyme source such as an immobilized material of the bacterial cells, a sonicate of the bacterial cells, a mechanically ground material of the bacterial cells, a crude enzyme extract obtained from the treated bacterial cells, and a purified enzyme obtained from the treated bacterial cells.

Among these, preferred examples include a concentrate of the above culture, a dried material of the culture, bacterial cells obtained by subjecting the culture to centrifugation, filtration, or the like, a dried material of the bacterial cells, a lyophilizate of the bacterial cells, a surfactant-treated material of the bacterial cells, a solvent-treated material of the bacterial cells, an enzyme-treated material of the bacterial cells, a material containing viable bacterial cells that retain the same function as the culture as the enzyme source such as an immobilized material of the bacterial cells, a sonicate of the bacterial cells, and a mechanically ground material of the bacterial cells, and most preferred examples include a concentrate of the above culture, a dried material of the culture, bacterial cells obtained by subjecting the culture to centrifugation, filtration, or the like, a dried material of the bacterial cells, a lyophilizate of the bacterial cells, a surfactant-treated material of the bacterial cells, a solvent-treated material of the bacterial cells, an enzyme-treated material of the bacterial cells, and a material containing viable bacterial cells that retain the same function as the culture as the enzyme source such as an immobilized material of the bacterial cells.

The concentration of the protein having NDST activity as the enzyme source is preferably 1 mg/L to 500 g/L, more preferably 1 mg/L to 300 g/L, and most preferably 1 mg/L to 5 g/L.

The concentration of heparosan which is the substrate is preferably 1 mg/L to 100 g/L, and more preferably 10 mg/L to 20 g/L.

The concentration of PAPS, which is the sulfate group donor, is preferably 0.1 to 500 mM, and more preferably 0.5 to 50 mM. In addition, PAPS is converted to 3′-phosphoadenosine-5′-phosphate (hereinafter also referred to as “PAP”) when used in an N-sulfation reaction, and therefore, an enzyme and/or a substrate for regenerating PAPS from PAP may be added. As the enzyme and/or the substrate for regenerating PAPS, ATP sulfurylase, adenosine 5′-phosphosulfate kinase, an ATP or ATP source, a sulfate ion source (WO2021/201282), 3′-phosphoadenosine 5′-phosphosulfate-sulfotransferase, p-nitrophenyl sulfate (pNPS) (WO2020/013346), and the like are exemplified.

Examples of the aqueous medium include water, buffers such as a phosphate, a carbonate, an acetate, a borate, a citrate, and Tris, alcohols such as methanol and ethanol, esters such as ethyl acetate, ketones such as acetone, and amides such as acetamide. In addition, the culture solution of the microorganism used as the enzyme source can be used as the aqueous medium.

The heparosan-derived compound produced in the aqueous medium can be quantified and collected by the methods described above in (alpha).

One embodiment of the method for producing a heparosan-derived compound of the invention may include at least one of the following steps in addition to the step (III) or the step (ii). The following steps are examples of the step when producing heparin from heparosan (WO2017/115674 and WO2017/115675).
a step of producing heparosan having a lower molecular weight by decomposing heparosan
a step of isomerizing a beta-D-glucuronic acid residue in heparosan to an alpha-L-iduronic acid (IdoA) residue which is an epimer
a step of sulfating a hydroxy group at the 2-position in a hexuronic acid residue (preferably alpha-L-iduronic acid residue) in heparosan
a step of sulfating a hydroxy group at the 6-position of an alpha-D-glucosamine residue in heparosan
a step of sulfating a hydroxy group at the 3-position of an alpha-D-glucosamine residue in heparosan

As described above, the present specification discloses the following matters.

{1} A method for producing a microorganism having N-deacetylation activity and N-sulfation activity, comprising:
a step (I) of modifying a nucleotide sequence of a DNA encoding N-deacetylase/N-sulfotransferase; and
a step (II) of introducing the DNA containing the nucleotide sequence modified in the step (I) into a microorganism in an expressible manner, wherein
the step (I) is a step of performing codon optimization according to the codon usage frequency of a biological species different from the microorganism.

{2} The method for producing a microorganism having N-deacetylation activity and N-sulfation activity according to above {1}, wherein the microorganism is a bacterium, and the biological species is budding yeast.

{3} The method for producing a microorganism having N-deacetylation activity and N-sulfation activity according to above {2}, wherein the DNA containing the modified nucleotide sequence is a DNA in (A-1) or (A-2):
(A-1) a DNA that contains the nucleotide sequence represented by SEQ ID NO: 6; or
(A-2) a DNA that contains a nucleotide sequence having 80% or more identity to the nucleotide sequence represented by SEQ ID NO: 6, and that encodes a protein having N-deacetylation activity and N-sulfation activity.

{4} A method for producing a heparosan-derived compound, comprising a step (III) of producing an N-deacetylated and N-sulfated heparosan-derived compound from heparosan in the presence of a microorganism having N-deacetylation activity and N-sulfation activity produced by the production method according to any one of above {1} to {3} or an extract thereof.

{5} The production method according to any one of above {1} to {4}, wherein the microorganism is a bacterium of the genus Escherichia.

{6} The production method according to above {5}, wherein the bacterium of the genus Escherichia is Escherichia coli.

{7} The production method according to above {4}, wherein the heparosan-derived compound is N-sulfated heparosan.

{8} A method for producing a microorganism having N-deacetylation activity and N-sulfation activity, comprising a step (i) of expressing any one protein selected from the group consisting of the following (B-1) to (B-3), (C-1) to (C-3), (D-1) to (D-3) and (E-1) to (E-3) in a microorganism:
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

{9} The method for producing a microorganism having N-deacetylation activity and N-sulfation activity according to above {8}, wherein
the step (i) is a step of introducing any one DNA selected from the group consisting of the following (b-1) to (b-3), (c-1) to (c-3), (d-1) to (d-3) and (e-1) to (e-3) into the microorganism in an expressible manner:
(b-1) a DNA that contains the nucleotide sequence represented by SEQ ID NO: 9;
(b-2) a DNA that encodes a protein containing the amino acid sequence represented by SEQ ID NO: 13;
(b-3) a DNA that contains a nucleotide sequence having 80% or more identity to the nucleotide sequence represented by SEQ ID NO: 9, and that encodes a protein having N-deacetylation activity and N-sulfation activity;
(c-1) a DNA that contains the nucleotide sequence represented by SEQ ID NO: 10;
(c-2) a DNA that encodes a protein containing the amino acid sequence represented by SEQ ID NO: 14;
(c-3) a DNA that contains a nucleotide sequence having 80% or more identity to the nucleotide sequence represented by SEQ ID NO: 10, and that encodes a protein having N-deacetylation activity and N-sulfation activity;
(d-1) a DNA that contains the nucleotide sequence represented by SEQ ID NO: 11;
(d-2) a DNA that encodes a protein containing the amino acid sequence represented by SEQ ID NO: 15;
(d-3) a DNA that contains a nucleotide sequence having 80% or more identity to the nucleotide sequence represented by SEQ ID NO: 11, and that encodes a protein having N-deacetylation activity and N-sulfation activity;
(e-1) a DNA that contains the nucleotide sequence represented by SEQ ID NO: 12; (e-2) a DNA that encodes a protein containing the amino acid sequence represented by SEQ ID NO: 16; and
(e-3) a DNA that contains a nucleotide sequence having 80% or more identity to the nucleotide sequence represented by SEQ ID NO: 12, and that encodes a protein having N-deacetylation activity and N-sulfation activity.

{10} A method for producing a heparosan-derived compound, comprising a step (ii) of producing an N-deacetylated and N-sulfated heparosan-derived compound from heparosan in the presence of a microorganism having N-deacetylation activity and N-sulfation activity produced by the production method according to above {8} or {9} or an extract thereof.

{11} The production method according to any one of above {8} to {10}, wherein the microorganism is a bacterium of the genus Escherichia.

{12} The production method according to above {11}, wherein the bacterium of the genus Escherichia is Escherichia coli.

{13} The production method according to above {10}, wherein the heparosan-derived compound is N-sulfated heparosan.

{14} A microorganism having N-deacetylation activity and N-sulfation activity, which expresses any one protein selected from the group consisting of the following (B-1) to (B-3), (C-1) to (C-3), (D-1) to (D-3) and (E-1) to (E-3):
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

### EXAMPLES

Hereinafter, the invention will be specifically described with reference to Examples, but the invention is not limited to the following Examples as long as the gist thereof is not exceeded.

### Analysis Example

The analysis of the N-deacetylation and N-sulfation reaction activities for heparosan was performed by the HPLC method as follows.

According to WO2018/048973, unsaturated disaccharide production by enzymatic digestion and an analysis by HPLC were performed. The obtained reaction solution was centrifuged, and the resulting supernatant was heated and maintained at 80 degree Celsius for 10 minutes to perform protein denaturation. The solution after protein denaturation was centrifuged, and the resulting supernatant was desalted by ultrafiltration using an Amicon Ultra centrifugal filter 3K device (Merck). The solution after desalting was subjected to a heparinase reaction solution [a composition composed of 50 mM ammonium acetate and 2 mM calcium chloride] containing 0.5 U/ml each of heparinase I, II, and III (Sigma), and enzymatically digested at 35 degree Celsius for 2 hours. The solution after enzymatic digestion was maintained at 95 degree Celsius for 15 minutes, thereby inactivating heparinase.

An unsaturated disaccharide analysis was performed by subjecting the solution after inactivation of heparinase to an analysis in a gradient elution mode with a mobile phase A [an aqueous solution containing 1.8 mM sodium dihydrogen phosphate and adjusted to pH 3.0 with phosphoric acid] and a mobile phase B [an aqueous solution containing 1.8 mM sodium dihydrogen phosphate and 1 M sodium perchlorate and adjusted to pH 3.0 with phosphoric acid] using general-purpose HPLC Prominence (Shimadzu Corporation) and a strong anion exchange column (spherisorb-SAX chromatography column, 4.0×250 mm, 5 micrometer, Waters Corporation).

An unsaturated disaccharide was detected by measuring an absorbance at 232 nm using a UV detector SPD-20A (Shimadzu Corporation). By comparison with an unsaturated disaccharide standard (Iduron Ltd.), the retention times of delta UA-GlcNAc, delta UA-GlcN, and delta UA-GlcNS were confirmed. Delta UA means 4,5 unsaturated uronic acid, GlcNAc means N-acetylglucosamine, GlcN means glucosamine, and GlcNS means N-sulfoglucosamine. By determining the area ratio of delta UA-GlcNS to the total area of the detected delta UA-GlcNAc, delta UA-GlcN, and delta UA-GlcNS, the N-deacetylase and N-sulfatase activities can be confirmed, and by determining the area ratio of delta UA-GlcN to the total area of delta UA-GlcNAc, delta UA-GlcN, and delta UA-GlcNS, the N-deacetylase activity can be confirmed. Hereafter, delta UA-GlcNAc is abbreviated as GlcNAc, delta UA-GlcN as GlcN, and delta UA-GlcNS as GlcNS.

### Example 1

### Production of Microorganism Expressing NDST (1)

### (1) Construction of Expression Plasmid for Wild-Type Rat NDST-1

A plasmid for expressing a rat-derived wild-type NDST gene (SEQ ID NO: 1) was produced by the following procedure.

By using a mouse liver-derived 1st strand cDNA purchased from Genostaff as a template, PCR was performed using primers composed of SEQ ID NOS: 2 and 3, thereby amplifying a rat-derived wild-type NDST gene. Subsequently, by using an expression vector pGEX-4T3 (GE Healthcare Life Sciences) as a template, PCR was performed using primers composed of SEQ ID NOS: 4 and 5. By using the obtained fragment and a DNA obtained by preparing the rat-derived wild-type NDST gene fragment prepared earlier, ligation was performed using the In-Fusion HD Cloning Kit (Takara Bio Inc.), thereby obtaining an expression plasmid pGEX-NDST (WT).

### (2) Construction of Expression Plasmid for Budding Yeast Codon-Optimized Rat NDST-1

A plasmid for expressing a budding yeast codon-optimized NDST gene (SEQ ID NO: 6) was produced by the following procedure.

By using a DNA synthesized by Eurofins as a template, PCR was performed using primers composed of SEQ ID NOS: 7 and 8, thereby amplifying a rat-derived wildtype NDST gene. Subsequently, by using an expression vector pGEX-4T3 (GE Healthcare Life Sciences) as a template, PCR was performed using primers composed of SEQ ID NOS: 4 and 5. By using the obtained fragment and a DNA obtained by preparing the rat-derived wild-type NDST gene fragment prepared earlier, a restriction enzyme treatment with BamHI and EcoRI was performed for each, and the resultant fragments were linked by ligation, thereby obtaining an expression plasmid pGEX-NDST (SC).

(3) Construction of NDST-1-Expressing Escherichia coli Strain Transfected with Plasmid

Escherichia coli Origami B (DE3) (Novagen) transformed with the constructed expression plasmid vector was selected with ampicillin, thereby obtaining an Escherichia coli strain expressing NDST.

### Example 2

### NDST Reaction Test (1)

### (1) Culturing Method

Escherichia coli Origami B (DE3) transformed with the expression plasmid was cultured overnight at 30 degree Celsius on an LB agar medium supplemented with ampicillin. The grown bacterial cells were inoculated into an LB liquid medium supplemented with ampicillin, and cultured overnight in a test tube at 30 degree Celsius with shaking, thereby obtaining a seed culture solution. The seed culture solution was further inoculated in an amount of 5 vol % into an LB liquid medium supplemented with ampicillin. This culture solution was cultured with shaking at 30 degree Celsius, and IPTG was added to a final concentration of 0.1 mM when the absorbance at 660 nm with a spectrophotometer reached 0.4 to 0.6, and induction culture was performed at 20 degree Celsius for 24 hours. After completion of the culturing, the bacterial cells were recovered and centrifuged at 9,000 rpm at 4 degree Celsius for 5 minutes. The bacterial cells were stored at −80 degree Celsius.

### (2) Reaction Method

The recovered Escherichia coli cells was suspended in an extraction buffer [0.1 M MES/NaOH (6.5), 20 mM MnCl₂, 5% (v/v) glycerol, and 1 mM PMSF] to give a wet weight of 140 g/L. The Escherichia coli suspension was disrupted by sonication on ice and centrifuged at 12,000 rpm at 4 degree Celsius for 5 minutes. The supernatant was recovered as an Escherichia coli extract solution and used in an enzymatic reaction.

### (3) Enzymatic Reaction

A substrate and the Escherichia coli extract solution were mixed to give [50 mM MES/NaOH (6.5), 10 mM MnCl₂, 1 g/L heparosan, 4.4 mM PAPS, and a 50% (v/v) Escherichia coli crude extract solution] in a 1.5 mL tube and allowed to react in an incubator at 30 degree Celsius for 24 hours. The reaction was stopped by a heat treatment at 80 degree Celsius for 10 minutes.

### (4) Reaction Test Results

The results of the reaction test are summarized in Table 1. GlcNAc indicates the presence of unreacted heparosan, GlcN indicates the presence of N-deacetylated heparosan, and GlcNS indicates the presence of N-sulfated heparosan.

TABLE 1

| | Ratio of disaccharides | | |
|---|---|---|---|
| | (%) | | |
| | GlcNAc | GlcN | GlcNS |
| pGEX-NDST(SC) | 39.5 | 0 | 60.5 |
| pGEX-NDST(WT) | 68.0 | 0 | 32.0 |

The pGEX-NDST (SC), in which NDST having been subjected to codon optimization according to the codon usage frequency of budding yeast was introduced, showed unexpectedly high N-deacetylation and N-sulfation reactions.

### Example 3

### Production of Microorganism Expressing NDST (2)

In each of the genes of human-derived NDST-1 to 4, continuous rare codons were changed to optimal codons for Escherichia coli, a nucleotide sequence at a site where a loop may be formed on mRNA was changed, a GC content was changed to be closer to that of Escherichia coli, and further, amino acids arranged on the surface of the tertiary structure were changed. Modified NDST-1 to 4 represented by SEQ ID NOS: 9 to 12, respectively, which are nucleotide sequences designed in this manner, were each synthesized. Each DNA sequence was cleaved with NdeI and BamHI and inserted into the BamHI-NdeI site of PET15b, thereby obtaining NDST1_pET15b, NDST2_pET15b, NDST3_pET15b, and NDST4_pET15b, respectively. The amino acid sequences of NDST-1 to 4 after modification are represented by SEQ ID NOS: 13 to 16, respectively.

### Example 4

### NDST Reaction Test (2)

Each plasmid was introduced into Escherichia coli Origami B (DE3), and Escherichia coli was cultured in LB medium at 22 degree Celsius. IPTG was added to a concentration of 1 mM when Escherichia coli was grown and the OD600 reached 0.8 to 1.0, and Escherichia coli was further cultured for 16 to 20 hours. Thereafter, the bacterial cells were collected and disrupted by ultrasonic waves, and the centrifugal supernatant was prepared as a cell extract solution. To a solution at pH 6.8 to 7.0 composed of 50 mM MES, 125 mM NaCl, and 10 mM MgCl₂, and containing this cell extract solution at 50%, the following substrate was added to give 2 mg/mL, and a reaction was allowed to proceed at 37 degree Celsius for 18 hours. In the case of the following (b) and (c), PAPS was added to the solution to give a final concentration of 5 mM.

The reaction was allowed to proceed under the following three conditions.
(a) Heparosan was used as the substrate and PAPS was not added.
(b) N-Deacetylated heparosan was used as the substrate and PAPS was added.
(c) Heparosan was used as the substrate and PAPS was added.

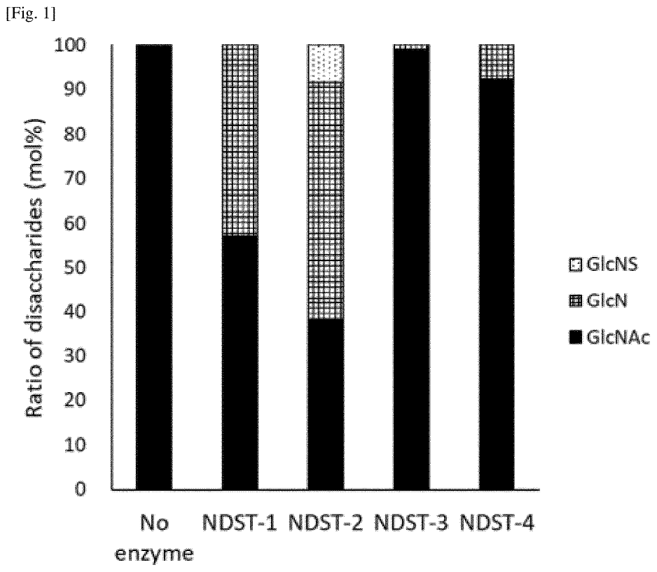

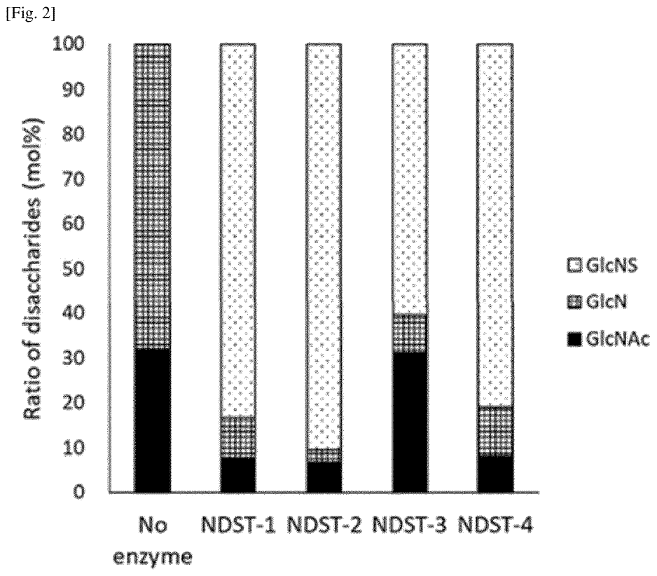

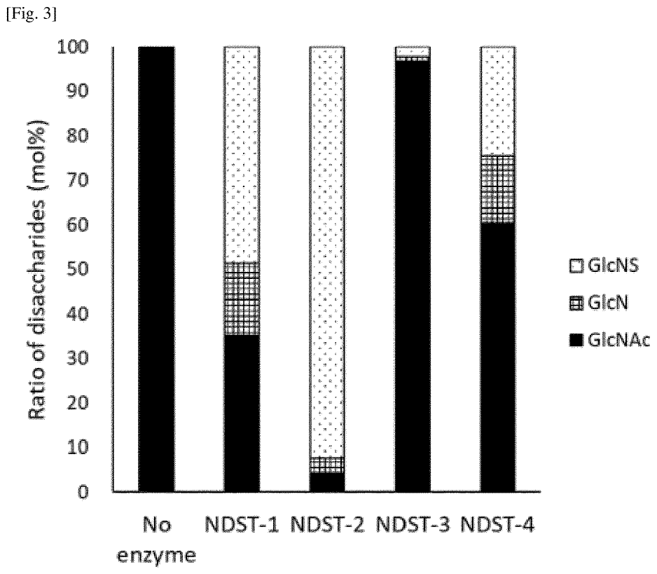

The reaction was stopped by a heat treatment at 95 degree Celsius for 10 minutes, and after decomposing the produced sugar with heparinase I, II, and III, the amounts of constituent disaccharides were analyzed by HPLC. The results in the case of the above (a) are shown in FIG. 1, the results in the case of the above (b) are shown in FIG. 2, and the results in the case of the above (c) are shown in FIG. 3. GlcNAc indicates the presence of unreacted heparosan, GlcN indicates the presence of deacetylated heparosan, and GlcNS indicates the presence of N-sulfated heparosan.

As shown in FIG. 1 and Table 2, in the case of (a) where heparosan was used as the substrate in the absence of PAPS, about 50% of GlcNAc was deacetylated with NDST1 and NDST-2. As shown in FIG. 2 and Table 2, in the case of (b) where the reaction was allowed to proceed using deacetylated heparosan as the substrate in the presence of PAPS, significant production of GlcNS was observed when using any enzyme, and the activities of NDST-1, 2, and 4 were high. As shown in FIG. 3 and Table 2, in the case of (c) where heparosan was used as the substrate in the presence of PAPS, GlcNS was produced with NDST-1, 2, and 4, and therefore, it was found that NDST-1, 2, and 4 have both N-deacetylation activity and N-sulfation activity. NDST-2 showed a GlcNS ratio of 85% or more and therefore was found to have particularly high N-deacetylation and N-sulfation activities as an enzyme expressed by the bacterium.

TABLE 2

| | | No | | | | |
|---|---|---|---|---|---|---|
| | | enzyme | NDST-1 | NDST-2 | NDST-3 | NDST-4 |
| (a) | GlcNAc | 99.8 | 57.2 | 38.2 | 98.8 | 92.4 |
| Heparosan | GlcN | 0.2 | 42.8 | 53.3 | 1.2 | 7.6 |
| only | GlcNS | 0 | 0 | 8.6 | 0 | 0 |
| (b) | GlcNAc | 31.9 | 7.6 | 6.8 | 31.2 | 8.1 |
| DeAc- | GlcN | 68.1 | 9 | 2.7 | 8.4 | 10.9 |
| Heparosan + | GlcNS | 0 | 83.3 | 90.5 | 60.4 | 81 |
| PAPS | | | | | | |
| (c) | GlcNAc | 99.8 | 35.2 | 4.2 | 96.9 | 60.4 |
| Heparosan + | GlcN | 0.2 | 16.2 | 3.5 | 0.9 | 14.9 |
| PAPS | GlcNS | 0 | 48.6 | 92.4 | 2.2 | 24.7 |

The present invention has been described in detail with reference to the specific aspects, but it is obvious for those skilled in the art that various changes and modifications can be made without departing from the spirit and scope of the invention. The present application is based on a US Patent Application (patent application Ser. No. 18/326,155) filed on May 31, 2023, which is incorporated by reference in its entirety. Also, all references cited herein are incorporated in their entirety.

### Sequence Listing Free Text

SEQ ID NO: 1: nucleotide sequence of rat-derived wild-type NDST gene
SEQ ID NO: 2: nucleotide sequence of primer F for amplifying rat-derived wild-type NDST gene
SEQ ID NO: 3: nucleotide sequence of primer R for amplifying rat-derived wild-type NDST gene
SEQ ID NO: 4: nucleotide sequence of primer F for amplifying pGEX-4T3
SEQ ID NO: 5: nucleotide sequence of primer R for amplifying pGEX-4T3
SEQ ID NO: 6: nucleotide sequence of budding yeast codon-optimized NDST gene
SEQ ID NO: 7: nucleotide sequence of primer F for amplifying budding yeast codon-optimized NDST gene
SEQ ID NO: 8: nucleotide sequence of primer R for amplifying budding yeast codon-optimized NDST gene
SEQ ID NO: 9: nucleotide sequence of modified NDST-1 gene
SEQ ID NO: 10: nucleotide sequence of modified NDST-2 gene
SEQ ID NO: 11: nucleotide sequence of modified NDST-3 gene
SEQ ID NO: 12: nucleotide sequence of modified NDST-4 gene
SEQ ID NO: 13: amino acid sequence of modified NDST-1
SEQ ID NO: 14: amino acid sequence of modified NDST-2
SEQ ID NO: 15: amino acid sequence of modified NDST-3
SEQ ID NO: 16: amino acid sequence of modified NDST-4

1. A method for producing a microorganism having N-deacetylation activity and N-sulfation activity, comprising:
a step (I) of modifying a nucleotide sequence of a DNA encoding N-deacetylase/N-sulfotransferase; and
a step (II) of introducing the DNA containing the nucleotide sequence modified in the step (I) into a microorganism in an expressible manner, wherein
the step (I) is a step of performing codon optimization according to the codon usage frequency of a biological species different from the microorganism.

2. The method for producing a microorganism having N-deacetylation activity and N-sulfation activity according to claim 1, wherein the microorganism is a bacterium, and the biological species is budding yeast.

3. The method for producing a microorganism having N-deacetylation activity and N-sulfation activity according to claim 2, wherein the DNA containing the modified nucleotide sequence is a DNA in (A-1) or (A-2):
(A-1) a DNA that contains the nucleotide sequence represented by SEQ ID NO: 6; or
(A-2) a DNA that contains a nucleotide sequence having 80% or more identity to the nucleotide sequence represented by SEQ ID NO: 6, and that encodes a protein having N-deacetylation activity and N-sulfation activity.

4. A method for producing a heparosan-derived compound, comprising a step (III) of producing an N-deacetylated and N-sulfated heparosan-derived compound from heparosan in the presence of a microorganism having N-deacetylation activity and N-sulfation activity produced by the production method according to claim 1 or an extract thereof.

5. The production method according to claim 1, wherein the microorganism is a bacterium of the genus Escherichia.

6. The production method according to claim 5, wherein the bacterium of the genus Escherichia is Escherichia coli.

7. The production method according to claim 4, wherein the heparosan-derived compound is N-sulfated heparosan.

8. A method for producing a microorganism having N-deacetylation activity and N-sulfation activity, comprising a step (i) of expressing any one protein selected from the group consisting of the following (B-1) to (B-3), (C-1) to (C-3), (D-1) to (D-3) and (E-1) to (E-3) in a microorganism:
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.

9. The method for producing a microorganism having N-deacetylation activity and N-sulfation activity according to claim 8, wherein
the step (i) is a step of introducing any one DNA selected from the group consisting of the following (b-1) to (b-3), (c-1) to (c-3), (d-1) to (d-3) and (e-1) to (e-3) into the microorganism in an expressible manner:
(b-1) a DNA that contains the nucleotide sequence represented by SEQ ID NO: 9;
(b-2) a DNA that encodes a protein containing the amino acid sequence represented by SEQ ID NO: 13;
(b-3) a DNA that contains a nucleotide sequence having 80% or more identity to the nucleotide sequence represented by SEQ ID NO: 9, and that encodes a protein having N-deacetylation activity and N-sulfation activity;
(c-1) a DNA that contains the nucleotide sequence represented by SEQ ID NO: 10;
(c-2) a DNA that encodes a protein containing the amino acid sequence represented by SEQ ID NO: 14;
(c-3) a DNA that contains a nucleotide sequence having 80% or more identity to the nucleotide sequence represented by SEQ ID NO: 10, and that encodes a protein having N-deacetylation activity and N-sulfation activity;
(d-1) a DNA that contains the nucleotide sequence represented by SEQ ID NO: 11;
(d-2) a DNA that encodes a protein containing the amino acid sequence represented by SEQ ID NO: 15;
(d-3) a DNA that contains a nucleotide sequence having 80% or more identity to the nucleotide sequence represented by SEQ ID NO: 11, and that encodes a protein having N-deacetylation activity and N-sulfation activity;
(e-1) a DNA that contains the nucleotide sequence represented by SEQ ID NO: 12;
(e-2) a DNA that encodes a protein containing the amino acid sequence represented by SEQ ID NO: 16; and
(e-3) a DNA that contains a nucleotide sequence having 80% or more identity to the nucleotide sequence represented by SEQ ID NO: 12, and that encodes a protein having N-deacetylation activity and N-sulfation activity.

10. A method for producing a heparosan-derived compound, comprising a step (ii) of producing an N-deacetylated and N-sulfated heparosan-derived compound from heparosan in the presence of a microorganism having N-deacetylation activity and N-sulfation activity produced by the production method according to claim 8 or an extract thereof.

11. The production method according to claim 8, wherein the microorganism is a bacterium of the genus Escherichia.

12. The production method according to claim 11, wherein the bacterium of the genus Escherichia is Escherichia coli.

13. The production method according to claim 10, wherein the heparosan-derived compound is N-sulfated heparosan.

14. A microorganism having N-deacetylation activity and N-sulfation activity, which expresses any one protein selected from the group consisting of the following (B-1) to (B-3), (C-1) to (C-3), (D-1) to (D-3) and (E-1) to (E-3):
(B-1) a protein that contains the amino acid sequence represented by SEQ ID NO: 13;
(B-2) a protein that contains an amino acid sequence having 80% or more identity to the amino acid sequence represented by SEQ ID NO: 13, and that has N-deacetylation activity and N-sulfation activity;
(B-3) a protein that contains an amino acid sequence in which one or several amino acid residues have been deleted, substituted, added, or inserted in the amino acid sequence represented by SEQ ID NO: 13, and that has N-deacetylation activity and N-sulfation activity;
(C-1) a protein that contains the amino acid sequence represented by SEQ ID NO: 14;
(C-2) a protein that contains an amino acid sequence having 80% or more identity to the amino acid sequence represented by SEQ ID NO: 14, and that has N-deacetylation activity and N-sulfation activity;
(C-3) a protein that contains an amino acid sequence in which one or several amino acid residues have been deleted, substituted, added, or inserted in the amino acid sequence represented by SEQ ID NO: 14, and that has N-deacetylation activity and N-sulfation activity;
(D-1) a protein that contains the amino acid sequence represented by SEQ ID NO: 15;
(D-2) a protein that contains an amino acid sequence having 80% or more identity to the amino acid sequence represented by SEQ ID NO: 15, and that has N-deacetylation activity and N-sulfation activity;
(D-3) a protein that contains an amino acid sequence in which one or several amino acid residues have been deleted, substituted, added, or inserted in the amino acid sequence represented by SEQ ID NO: 15, and that has N-deacetylation activity and N-sulfation activity;
(E-1) a protein that contains the amino acid sequence represented by SEQ ID NO: 16;
(E-2) a protein that contains an amino acid sequence having 80% or more identity to the amino acid sequence represented by SEQ ID NO: 16, and that has N-deacetylation activity and N-sulfation activity; and
(E-3) a protein that contains an amino acid sequence in which one or several amino acid residues have been deleted, substituted, added, or inserted in the amino acid sequence represented by SEQ ID NO: 16, and that has N-deacetylation activity and N-sulfation activity.


SEQUENCE LISTING (16 sequences)

SEQ ID NO: 1 - DNA, 2520 bp, Rattus norvegicus
  source 1..2520: mol_type = genomic DNA
atgctcgagccctcggcagatgcttctgagtccgactgcggggacccaccacctgtcgcccctagccgtctcctgccaatcaagcctgtgcaggcggtcgccccttctcgaacagacccgctggtgctggtatttgtggagagcctctattcacagctgggccaggaggtggtggccatcctggaatccagtcgcttcaagtaccgaacagaaattgcaccggggaagggggacatgcccacactcacagacaagggccgaggccgcttcgccctcatcatctatgagaacatcctcaagtatgtcaacctggatgcctggaaccgggagctgctggacaagtactgtgtggcctacggcgtgggcatcattggcttcttcaaggccaatgagaacagcctgctgagtgcacagctcaaaggcttccctcttttcctgcattcgaacctgggcttgaaagactgcagcatcaaccccaagtccccactgctgtacgtgacacggcccagtgaggtagagaaaggtgtgctgcccggagaggactggacggtgttccagtctaaccactctacctatgagccagtgctgctggccaagacgcgctcctctgagtccatcccacacctgggcgcagatgccggcctgcatgctgccctgcacgctactgtggtccaggacctgggcctccatgacggcattcagcgtgtgctgtttggcaacaacctcaacttttggctgcataagctcgtcttcgtggacgctgtggccttcctcacagggaagcgcctctcactgcctttggaccgatacatcctggtggacattgatgacatttttgtaggcaaggagggcacacgcatgaaggtggaggatgtgaaggccctgtttgatacacagaatgaacttcgtacacatatcccaaacttcaccttcaacctgggctactcagggaaattcttccacacaggtaccgatgctgaggatgctggggacgacctgctgctgtcctatgtgaaagagttctggtggttcccccacatgtggagccatatgcaaccccacctcttccacaaccagtctgtgctggctgagcagatggccctgaacaagaagttcgctgtcgagcacggcattcccacagatatggggtatgcagtggcaccccaccactctggtgtgtaccctgtgcatgtgcagctgtatgaggcctggaagcaagtgtggaacatccgtgtgaccagcacagaggagtacccgcatctgaagcctgcccgttaccgccgtggcttcatccacaatggcatcatggtcctccctcggcagacctgtggtctctttacacacaccatcttctacaacgagtaccctggaggctccagtgagctggacaagatcatcaatgggggcgagctctttcttactgtgctcctcaatcctatcagcgtcttcatgacacacttatccaactatggaaatgaccgcctgggactgtacaccttcaagcacctggtgcgcttcctgcactcctggaccaacctgaggctgcagacgctgccccctgtgcagctggcccagaagtacttccagatcttttctgaggagaaggacccactttggcaggatccctgtgaggacaaacgccacaaagacatctggtctaaggagaagacatgtgatcgcttcccaaagctgctcatcattggcccccagaaaacaggcaccacagccctctacctgttcctgggcatgcaccccgacctcagcagcaactaccccagctccgagacctttgaggagatccagttttttaatggccacaactatcacaaaggcatcgactggtacatggaattcttccctattccctccaacaccacctctgacttctactttgaaaaaagtgccaactactttgattcagaagtggcaccacggcgagcagctgccctattgcccaaggccaaggttctcaccatcctcatcaatccagccgaccgggcttactcctggtaccagcaccagcgggcccatgatgacccggtggccctaaagtacaccttccatgaggtgatcacagctggccctgacgcatcctcaaagctgcgtgccctccagaaccgatgcctggtccccggctggtatgccactcatattgaacgctggctcagcgcctttcatgccaaccagatcctggtcttggatggcaaactgctgcgaacagaacctgccaaagtgatggacacagtgcagaaattcctcggggtgaccagcacggttgactaccataaaaccttggcgtttgacccaaagaaaggattttggtgccagctgctcgaaggaggaaaaaccaagtgtctgggaaaaagcaagggacggaaatatccagagatggacctggattcccgagccttcctaaaggattactaccgggaccacaacattgagctctctaagctgctgtataagatgggccagacactgcccacctggctgcgggaagacctccagaacaccaggtag

SEQ ID NO: 2 - DNA, 33 bp, synthetic construct
  source 1..33: mol_type = other DNA
gttccgcgtggatccatgctcgagccctcggca

SEQ ID NO: 3 - DNA, 35 bp, synthetic construct
  source 1..35: mol_type = other DNA
tcgacccgggaattcctacctggtgttctggaggt

SEQ ID NO: 4 - DNA, 20 bp, synthetic construct
  source 1..20: mol_type = other DNA
gaattcccgggtcgactcga

SEQ ID NO: 5 - DNA, 20 bp, synthetic construct
  source 1..20: mol_type = other DNA
ggatccacgcggaaccagat

SEQ ID NO: 6 - DNA, 2517 bp, synthetic construct
  source 1..2517: mol_type = other DNA
ttggaaccatctgctgatgcctcagaatctgattgtggtgatcctccgccagtagctccgagtcgcctgttgccaattaaacccgtacaagctgttgcaccatcaagaactgatccgctagtccttgtatttgtggaatctctatactcacaattaggtcaagaagtcgttgctattctggagagttcaagattcaaataccgtacggaaattgcacctggcaaaggcgatatgccaacattgacggataaaggtagaggtagatttgccttaataatttacgagaatatcctaaaatacgtgaatttggatgcgtggaatagagaattgcttgataagtactgtgtagcatacggtgtcggaatcattggcttcttcaaagcaaatgagaactcgttattgtcagcccaattaaaaggctttcctcttttcttgcactcaaatttgggtcttaaggattgctcaattaacccaaaatctcccttgctttatgttactagacccagcgaagtcgagaaaggagtgctgcctggtgaagattggaccgtatttcagtctaatcactctacctatgagcctgtgttgctcgctaaaacaagatcgtccgaatcgataccccatctcggtgcagatgctggtttacatgccgctttgcatgctacagttgtacaggatttaggtctgcatgacggtattcaacgggtcttgtttgggaacaacttaaatttctggttgcataagttggtcttcgttgatgcagtagcttttctgactgggaaaagactcagtttaccactggatagatatatccttgttgacattgatgacattttcgttggcaaagaaggtaccaggatgaaagttgaggatgtgaaagcactattcgatactcaaaacgagttgcgcactcacataccgaactttacatttaacttaggctattccggtaagttctttcacactggtacagatgcagaagacgcaggcgatgatttgttgttgagctatgtcaaggaattttggtggtttcctcatatgtggtcccatatgcaaccccatttgtttcataaccagtcagtcctagctgagcaaatggcgcttaacaaaaaatttgcagttgaacacggtatccctactgacatgggctatgcagttgcacctcatcatagtggtgtttatcccgttcatgttcagttatatgaggcctggaaacaagtttggaatatacgtgttacttctacggaagaatatccgcatttaaaacctgcgagatacaggagagggtttatccataatgggatcatggtacttccaagacagacatgtgggttatttacacacactatcttttacaatgaatatccaggaggaagctcagaactggacaagatcataaatggaggagaattattccttacggtgttattaaatccaatttcggtcttcatgacacatttgtccaactacggaaatgaccgattaggattatatacctttaagcatttggtgaggtttctgcattcatggactaacttaaggctacaaaccttacctccagtccaattagctcagaaatacttccagattttttccgaggaaaaggacccactttggcaagatccatgcgaagataagcgtcacaaggacatttggagtaaggaaaaaacctgtgataggttccccaagttgttaataataggaccacaaaagactggaacgactgccttgtatctctttctaggaatgcatccagacttgtctagcaattacccttccagtgaaacatttgaggagatacagttctttaatggtcataactatcataagggtatagattggtacatggaatttttcccaattccatctaataccacatctgacttctactttgaaaaatctgctaactacttcgattctgaagttgcccctagaagagctgcagcgttacttcccaaagccaaagtgctaaccatactcattaatcctgctgacagagcttatagctggtatcaacaccaacgtgcccatgatgatccggttgctctcaaatatacttttcacgaagtaatcacagcgggtcctgatgcatcgtccaaacttagggctttgcaaaatcgttgcttagttccaggttggtatgctactcacattgaacgatggctgtccgcatttcatgccaaccagattctagtgttagacggcaagttactaagaacggaacctgcgaaggtaatggataccgttcaaaagttccttggtgttacatctaccgtggactaccacaagactctagcttttgatccaaaaaagggtttctggtgtcaactattggaaggcggtaaaacaaagtgtttgggtaaaagtaaggggaggaaatatccagagatggatttggactcaagagcctttctaaaggactattatagggatcacaatattgaactgagtaaattactgtacaaaatgggtcaaacgttaccaacatggttgagagaagacctacaaaatactcggtaa

SEQ ID NO: 7 - DNA, 34 bp, synthetic construct
  source 1..34: mol_type = other DNA
gttccgcgtggatccttggaaccatctgctgatg

SEQ ID NO: 8 - DNA, 35 bp, synthetic construct
  source 1..35: mol_type = other DNA
gtcgacccggaattcttaccgagtattttgtaggt

SEQ ID NO: 9 - DNA, 2457 bp, synthetic construct
  source 1..2457: mol_type = other DNA
agccgtctgctgccgctgaagccggtgcaagcggcgaccccgagccgtaccgacccggtggttctggtgttcgttgagagcctgtacagccagctgggtcagcaaatcgttgcgattctggagagcaaccgttttaagtatcgtaccgaaattgcgccgggtaaaggtgacatgccgccgctgaccgataagggtcgtggccgttacgcgctgatcatttatgagaacattctgaaatacgtgaacctggacgcgtggaaccgtgaactgctggataagtactgccgtgcgtatggtgttggcatcattggcttctttaaagcgaacgaaaacagcctgctgagcgcgcaactgaagggtttcccgctgtttctgcacagcaacctgggcctgaaggactgcagcatcaacccgaaaagcccgctgctgcgtattacccgtccgagcgaggtgtggaaaggtgttctgccgggcgaagattggaccgtgttccagagcaaccacagcacctacgagccggttctgctggcgaagacccgtagcagcgaaagcatcccgcatctgggtgcggatgcgggtctgcatgcggcgctgcacatcaccgtggttctggacctgggtctgcacgatggcattcaacgtgtgctgttcggtaacaacctgaacttttggctgcacaaactggtgttcctggatgcggttgcgtttctgacccatggccgtctgagcctgccgctggaccgttatatcctggtggatatcgacgatattttcgttggtaaagagggtacccgtatgaaggtggaagacgttaaagcgctgctggatacccagaacgagctgcgtaaactgattccgaacttcacctttaacctgggttttagcggcaagttctttcacaccggtaccgacgaggaaaacaagggcgacgatctgctgctgaaacacgtggatgaattctggtggtttccgcacatgtggagccacatgcaaccgcacctgttccacaaccagagcgttctggcggagcaaatggcgctgaacaagcagtttgcgaaagaacacggtatcccgaccgacatgggttatgcggttgcgccgcaccacagcggtgtgtacccggttcacgagcaactgtatgaagcgtggaagcaagtgtggaacattaaagttaccagcaccgaggaatacccgcacctgaaaccggcgcgttatcgtcgtggtttcatccacaacggcattatggtgctgccgcgtcagacctgcggtctgttcacccacaccatcttttacaacgagtatccgggtggcccggaggaactggacaagagcattttcggtggcgaactgtttctgaccgttctgctgaacccgatcagcattttcatgacccacctgagcaactacggcaacgatcgtctggcgctgtataccttcaagcacctggtgaaatttctgcaaagctggaccaacctgcgtctgcaaaccctgccgccggttcagctggcgcaaaaatacttccaaatctttccggaggaaaaggacccgctgtggcagaacccgtgcgaggacaagcgtcacaaagatatttggagcaaggaaaaaacctgcgatcgtttcccgaagctgctgatcattggtccgcaaaaaaccggcaccaccgcgctgtacctgttcctgagcatgcacccggacctgatcagcaactatccgagcccggagacctttgaggaaattcagttctttaacggtaacaactaccacaaaggcatcgattggtatatgaacttctttccgattccgagcaacaccaccagcgacttctactttgagaagagcgcgaactatttcgatagcgaagtggcgccgcgtcgtgcggcggcgctgctgccgaaggcgaaaatcctgaccatcctgattaacccggcggaccgtgcgtacagctggtatcagcaccaacgtgcgcacaacgatccggtggcgctgaaatacacctttcacgaggttatcaccgcgggtagcgatgctccgccggaactgcgttggctgcagaaccgttgcctgctgccgggctggtatgcgacccacatcgagcgttggctgagctactatcacccgagccaaattctggtgctggacggtaaactgctgcgtaccgagccggcgaaggtgatggatgaagttcagaaattcctgggtgttaccaacatcattgactacagcaagcacctgaccttcgatccgaagaaaggcttttggtgccagctgctggaaggtggcaagaccaaatgcctgggcaagagcaaaggccgtaagtatccgccgatggacgaggatagccgtaagtttctgaaagactactatcgtgatcacaacatcgaactgagcaagctgctgcacaaaatgggtcaaccgctgccgacctggctgcgtgaggaactgcagaacacccgttaa

SEQ ID NO: 10 - DNA, 2454 bp, synthetic construct
  source 1..2454: mol_type = other DNA
gcgcgtccgccggttccgccgcgtccgccgcgtccgccggagaccgcgcgtaccgaaccggtggttctggtgttcgttgagagccagtacagccaactgggtcaggagatcgttgcgattctggaagcgctgcgttttaagtatcacaccgtgattgcgccgggtaaaggtgacatgccgccgctgaccgacaacgatcgtggccgttacgtgctgatcattttcgagaacctgctgaagtatgttaacctggacaaatggagccgtgagctgctggataagtactgccgtgaatatggtgttggcatcattggtttctttcgtgcgaccgaaaacagcctgctgagcgcgcaactgaaaggtttcccgctgtttctgcacaccaacctgggcctgcgtgattatcaagtgaacccgagcgcgccgctgctgcacatcacccgtccgagccagctggtgccgggtccgctgccgggtgacgattggaccgttttccagagcaaccacagcacctatgagccggtgctgctggcgagcctgcgtccggcggaaccgccggtgccgggtccggttccgcgtcgtgcgcgtctgccgaccgtggttctggacctgggtctgcacgatggcattcagcgtgttctgttcggccacgacctgagcttttggctgcacaagctgctgttcgtggatgcgatcgcgtacctgacccacaaacgtctgagcctgccgctggaccgttatattctggtggatatcgacgatatttttgttggcaagccgggcacccgtatgaagaaagaggacgttgaagcgctgctggatacccaaaacaaactgcgtaccctgatcccgaacttcacctttaacctgggtttcagcggcaagttttaccacaccggtaccgacgaggaagataaaggcgacgatatgctgctgaagcaccgtaaagagttctggtggtttccgcacatgtggagccacatgcagccgcacctgttccacaacgagagcgtgctggttgaacaaatgcgtctgaacaagcagtttgcgctggaacacggtattccgaccgacatgggttatgcggtggcgccgcaccacagcggtgtgtacccggttcacgagcaactgtatgaagcgtggaagaaagtgtggggcattcaggttaccagcaccgaggaatacccgcacctgaaaccggcgcgttatcgtcgtggtttcatccacaacggcattatggttctgccgcgtcagacctgcggtctgttcacccacaccatcttttacaacgagtatccgggtggcccggaggaactggaccgtagcattaacggtggcgaactgttcatgaccgtgctgctgaacccgatcagcatttttatgacccacctgagcaactacggcaacgatcgtctggcgctgtataccttcgaaagcctggttcgttttatccaatgctggaccaacctgcgtctgcaaaccctgccgccggtgcaactggcgcagaagtacttcgagctgtttccggaggaacgtgacccgctgtggcaaaacccgtgcgacgataagcgtcacaaagacatttggagcaaggaaaaaacctgcgatcgtctgccgaagttcctgatcgttggtccgcagaaaaccggcaccaccgcgatttatttctttctgagcctgcatccggcggtgaccagcaacttcccgagcccgaagacctttgaggaaatccagttctttaacagcccgaactaccacaaaggtattgactggtatatggatttctttccggttccgagcaacagcagcaccgacttcctgtttgagaagagcgcgacctacttcgatagcgaagtggcgccgcgtcgtgttgcggcgctgctgccgaaggcgaaaatcattgtggttctgatcaacccggcggaccgtgcgtacagctggtatcagcaccaacgtgcgcacggtgatccggtggcgctgaactacagcttttatcaagttattaccgcgagcgacgatagcccgaaagagctgcgttggctgcagaaccgttgcctgctgccgggttggtatgcgacccacctggaacgttggctgacctactatccgagcagccagctgctgatcgttgatggtcagcaactgcgtaccaacccggcggcggtgatggagaagatccaaaaattcctgggtattaccccgtttctgaactatagccagaccctgcgtttcgatccgaagaaaggcttttggtgccaactgctggaaggtggcaagaccaaatgcctgggcaagagcaaaggccgtaagtacccgccgatggacgaggaaagccgtaaattcctgaccgattattttcgtgagcacaacatcgaactgagcaaactgctgcaccgtctgggccaaccggtgccgacctggctgcgtgaggaactgcagcacagcagcctgggc

SEQ ID NO: 11 - DNA, 2445 bp, synthetic construct
  source 1..2445: mol_type = other DNA
taccagctgatggaagtgaaagcgatgaagctgttcgacgcgagccgtaccgatccggtggttctggtgtttgttgagagccagtacagccaactgggtcaggatatcattgcgatcctggaaagcaaccgtttccaatatcacatcgagattgcgccgggtaaaggcgacctgccggttctgatcgataagatgaagggcaagtacatcctgatcatctacgaaaacatcctgaagtacatcaacatggacagctggaaccgtagcctgctggataagtactgccgtgaatatggtgtgggcatcattggtttccacaagaccagcgagaaaagcgttcaaagctttcagctgaaaggtttcccgtttagcatctatggcaacctggcggtgaaggactgctgcatcaacccgcacagcccgctgattcgtgttaccaaaagcagcaagctggaaaaaggtagcctgccgggcaccgattggaccgtgttccaaatcaaccacagcgcgtaccagccggtgatttttgcgaaagttaagaccccggagaacctgagcccgagcatcagcaagggtgcgttctatgcgaccgtgatccacgacctgggtctgcacgatggcattcagcgtgttctgttcggcaacaacctgaacttttggctgcacaaactgatcttcattgacgcgatcagctttctgagccacaagcgtctgaccctgccgctggaccgttacattctggtggatatcgacgatattttcgttggtaaagagggtacccgtatgaacaccgacgatgttaaagcgctgctggacacccaaaacctgctgcgtaagcagatcaccaacttcacctttaacctgggtttcagcggcaaattttatcacaccggtaccgacgaggaagatgaaggcgacgatctgctgctgaaccacgtggatgagttctggtggtttccgcacatgtggagccacatgcagccgcacctgttccacaacgagagcagcctggttgaacaaatgcgtctgaacaagcagtttgcgctggaacacggtatcccgaccgacatgggttatgcggtggcgccgcaccacagcggtgtttacccggtgcacgttcagctgtatgaggcgtggaagaaagtgtggaacattaaagttaccagcaccgaggaatacccgcacctgaagccggcgcgttatcgtcgtggtttcatccacaacaacattatggtgctgccgcgtcaaacctgcggcctgttcacccacaccatcttttacaaagagtatccgggtggcccgaaggaactggacaaaagcattcacggtggcgaactgttctttaccgtggttctgaacccgatcaacatttttatgacccacctgagcaactacggtaacgatcgtctgggcctgtataccttcgagaacctggcgaactttctgcagagctggaccaacctgcgtctgcagaccctgccgccggtgcagctggcgcacaagtacttcgaactgtttccggaccaaaaagatccgctgtggcagaacccgtgcgacgataagcgtcacaaagacatctggagcaaagagaagacctgcgatcgtctgccgaagttcctggtgattggtccgcagaaaaccggcaccaccgcgctgtacctgttcctggttatgcacccgagcatcctgagcaacagcccgagcccgaagaccttcgaggaagttcaattctttaaccgtaacaactaccaccgtggtattgactggtatatggatttctttccggtgccgagcaacgttaccaccgatttcctgtttgagaagagcgcgaactactttcacagcgaggaagcgccgaagcgtgcggcggcgctggtgccgaaagcgaagatcattaccatcctgattaacccggcggaccgtgcgtacagctggtatcagcaccaacgtagccacaacgatccggcggcgctgaaatacagcttctatgaagttatcaccgcgggtccggatgcgccgaaggagctgcgttggctgcaaaaccgttgcctgctgccgggttggtatgcgcagcacattgaacgttggctgcaatatttcccgccgagccagctgctgatcattgacggtcagcaactgcgtaccgacccggcgaccgtgatggatgaggttcaaaaatttctgggcgttctgccgcactacaactatagccaggcgctgaccttcgatagccacaagggtttttggtgccaactgctggaggaaggcaaaaccaagtgcctgggtaaaagcaagggccgtaagtacccgccgatggacgaggatagccgtaaattcctgagcgaatactatcgtgaccacaacgtggagctgagcaagctgctgcacaagctgggtcagccgctgccgagctggctgcgtcaagagctgcagaaagttcgttaa

SEQ ID NO: 12 - DNA, 2442 bp, synthetic construct
  source 1..2442: mol_type = other DNA
taccgtagcatggaactgaagaccgtgaaaccgatcgacaccagcaagaccgatccggtggttctgctgttcgttgaaagccagtacagccaactgggtcaggacatcattgcgattctggaggcgaaccgttttcagtatcacctggtgatcgcgccgggtaaaggcgacattccgccgctgaccgataacggcaagggcaaatacatcctggtgatttatgagaacatcctgaagtatgttaacatggatagctggaaccgtgagctgctggaaaaatactgccgtgaatatagcgttagcatcattggtttccacaaggcgaacgagaacagcctgccgagcacccaactgaaaggcttcccgctgttcctgtttaacaacctggcgctgaaggactgctttgtgaacccgcagagcccgctgctgcacatcaccaaggcgccgaaagtttggtggggtccgctgccgggcgaagattggaccattttccaatacaaccacagcacctatcagccggtgctgctggcggagctgcaaaccgaaaagagcctgagcagcctgagcagcaaaaccctgtttgcgaccgtgatcctggacctgggtctgcacgatggcattcagcgtgttctgttcggtaacaacctgaacttttggctgcacaagctgatcttcctggacgcgattagctttctgagcggtggccgtctgagcctgagcctggaccgttacatcctggtggatatcgacgatattttcgttggtaaagagggcacccgtatgaacgtggaagatgttaaggcgctgctggagacccaaaacaaactgcgtacccaggttaccaacttcacctttaacctgggtttcagcggcaaattttatcacaccggtaccgacgaggaagatgaaggcgacgatatgctgctgcgtcacatcgacgagttctggtggtttccgcacatgtggagccacatgcaaccgcacctgttccacaacgagagcagcctggtggaacaaatgctgctgaacaagcagtttgcgaaagaacacggtattccgaccaacatgggttatgcggttgcgccgcaccacagcggtgtgtacccggttcacatccagctgtatgaggcgtggaagaaagtgtggggcattaaggttaccagcaccgaggaatacccgcacctgaaaccggcgcgttatcgtcgtggtttcatccacaacggcattatggtgctgccgcgtcaaacctgcggtctgttcacccacaccatcttttacaaggaatatccgggtggcccggaggaactggacaaaagcatccacggtggcgagctgttccagaccattctgctgaacccgatcagcatttttatgacccacctgagcaactacggcaacgatcgtctggcgctgtataccttcgtgaacctggttaactttatccagagctggaccaacctgaagctgcaaaccctgccgccggttcagctggcgcacaagtacttccaactgtttccggaacagaaagacccgctgtggcaaaacccgtgcgacgataagcgtcacaaagacatctggagccgtgagaagacctgcgatcacctgccgaaattcctggtgattggtccgcagaaaaccggcaccaccgcgctgtacctgttcctgctgatgcacccgagcatcattagcaacctgccgagcccgaagaccttcgaggaagttcagttctttaacggtaacaactaccacaaaggcatcgactggtatatggatttctttccgaccccgagcaacaccaccagcgacttcctgtttgagaagagcgcgaactactttcacagcgaggaagcgccgcgtcgtgcggcggcgctgctgccgaaggcgaaaatcattaccatcctgattgacccggcggatcgtgcgtacagctggtatcagcaccaacgtagccacaacgatccggcggcgctgaactacagcttctatgaagtgatcaccaccggtcactgggcgccgaaggagctgaaaaccctgcaaaaccgttgcctgctgccgggttggtatgcgacccacattgaacgttggctgacctatttccacccgagccagctgctgatcattgacggtcagcaactgcgtagcgacccggcgaccgtgatggatgaggttcaaaagtttctgggtgttaccccgcgttacaactatagccaggcgctgaccttcgatccgcaaaaaggcttttggtgccagctgctggaaggtggcaagaccaaatgcctgggcaagagcaaaggccgtaagtacccgccgatggacccggaaagccgtaaattcctgagccgttactatcgtgatcacaacgtggagctgagcaaactgctgcatcgtctgggtcagccgctgccgagctggctgcgtcaagagctgcagaaagttcgttaa

SEQ ID NO: 13 - protein, 818 aa, synthetic construct
SRLLPLKPVQAATPSRTDPVVLVFVESLYSQLGQQIVAILESNRFKYRTEIAPGKGDMPPLTDKGRGRYALIIYENILKYVNLDAWNRELLDKYCRAYGVGIIGFFKANENSLLSAQLKGFPLFLHSNLGLKDCSINPKSPLLRITRPSEVWKGVLPGEDWTVFQSNHSTYEPVLLAKTRSSESIPHLGADAGLHAALHITVVLDLGLHDGIQRVLFGNNLNFWLHKLVFLDAVAFLTHGRLSLPLDRYILVDIDDIFVGKEGTRMKVEDVKALLDTQNELRKLIPNFTFNLGFSGKFFHTGTDEENKGDDLLLKHVDEFWWFPHMWSHMQPHLFHNQSVLAEQMALNKQFAKEHGIPTDMGYAVAPHHSGVYPVHEQLYEAWKQVWNIKVTSTEEYPHLKPARYRRGFIHNGIMVLPRQTCGLFTHTIFYNEYPGGPEELDKSIFGGELFLTVLLNPISIFMTHLSNYGNDRLALYTFKHLVKFLQSWTNLRLQTLPPVQLAQKYFQIFPEEKDPLWQNPCEDKRHKDIWSKEKTCDRFPKLLIIGPQKTGTTALYLFLSMHPDLISNYPSPETFEEIQFFNGNNYHKGIDWYMNFFPIPSNTTSDFYFEKSANYFDSEVAPRRAAALLPKAKILTILINPADRAYSWYQHQRAHNDPVALKYTFHEVITAGSDAPPELRWLQNRCLLPGWYATHIERWLSYYHPSQILVLDGKLLRTEPAKVMDEVQKFLGVTNIIDYSKHLTFDPKKGFWCQLLEGGKTKCLGKSKGRKYPPMDEDSRKFLKDYYRDHNIELSKLLHKMGQPLPTWLREELQNTR

SEQ ID NO: 14 - protein, 818 aa, synthetic construct
ARPPVPPRPPRPPETARTEPVVLVFVESQYSQLGQEIVAILEALRFKYHTVIAPGKGDMPPLTDNDRGRYVLIIFENLLKYVNLDKWSRELLDKYCREYGVGIIGFFRATENSLLSAQLKGFPLFLHTNLGLRDYQVNPSAPLLHITRPSQLVPGPLPGDDWTVFQSNHSTYEPVLLASLRPAEPPVPGPVPRRARLPTVVLDLGLHDGIQRVLFGHDLSFWLHKLLFVDAIAYLTHKRLSLPLDRYILVDIDDIFVGKPGTRMKKEDVEALLDTQNKLRTLIPNFTFNLGFSGKFYHTGTDEEDKGDDMLLKHRKEFWWFPHMWSHMQPHLFHNESVLVEQMRLNKQFALEHGIPTDMGYAVAPHHSGVYPVHEQLYEAWKKVWGIQVTSTEEYPHLKPARYRRGFIHNGIMVLPRQTCGLFTHTIFYNEYPGGPEELDRSINGGELFMTVLLNPISIFMTHLSNYGNDRLALYTFESLVRFIQCWTNLRLQTLPPVQLAQKYFELFPEERDPLWQNPCDDKRHKDIWSKEKTCDRLPKFLIVGPQKTGTTAIYFFLSLHPAVTSNFPSPKTFEEIQFFNSPNYHKGIDWYMDFFPVPSNSSTDFLFEKSATYFDSEVAPRRVAALLPKAKIIVVLINPADRAYSWYQHQRAHGDPVALNYSFYQVITASDDSPKELRWLQNRCLLPGWYATHLERWLTYYPSSQLLIVDGQQLRTNPAAVMEKIQKFLGITPFLNYSQTLRFDPKKGFWCQLLEGGKTKCLGKSKGRKYPPMDEESRKFLTDYFREHNIELSKLLHRLGQPVPTWLREELQHSSLG

SEQ ID NO: 15 - protein, 814 aa, synthetic construct
YQLMEVKAMKLFDASRTDPVVLVFVESQYSQLGQDIIAILESNRFQYHIEIAPGKGDLPVLIDKMKGKYILIIYENILKYINMDSWNRSLLDKYCREYGVGIIGFHKTSEKSVQSFQLKGFPFSIYGNLAVKDCCINPHSPLIRVTKSSKLEKGSLPGTDWTVFQINHSAYQPVIFAKVKTPENLSPSISKGAFYATVIHDLGLHDGIQRVLFGNNLNFWLHKLIFIDAISFLSHKRLTLPLDRYILVDIDDIFVGKEGTRMNTDDVKALLDTQNLLRKQITNFTFNLGFSGKFYHTGTDEEDEGDDLLLNHVDEFWWFPHMWSHMQPHLFHNESSLVEQMRLNKQFALEHGIPTDMGYAVAPHHSGVYPVHVQLYEAWKKVWNIKVTSTEEYPHLKPARYRRGFIHNNIMVLPRQTCGLFTHTIFYKEYPGGPKELDKSIHGGELFFTVVLNPINIFMTHLSNYGNDRLGLYTFENLANFLQSWTNLRLQTLPPVQLAHKYFELFPDQKDPLWQNPCDDKRHKDIWSKEKTCDRLPKFLVIGPQKTGTTALYLFLVMHPSILSNSPSPKTFEEVQFFNRNNYHRGIDWYMDFFPVPSNVTTDFLFEKSANYFHSEEAPKRAAALVPKAKIITILINPADRAYSWYQHQRSHNDPAALKYSFYEVITAGPDAPKELRWLQNRCLLPGWYAQHIERWLQYFPPSQLLIIDGQQLRTDPATVMDEVQKFLGVLPHYNYSQALTFDSHKGFWCQLLEEGKTKCLGKSKGRKYPPMDEDSRKFLSEYYRDHNVELSKLLHKLGQPLPSWLRQELQKVR

SEQ ID NO: 16 - protein, 813 aa, synthetic construct
YRSMELKTVKPIDTSKTDPVVLLFVESQYSQLGQDIIAILEANRFQYHLVIAPGKGDIPPLTDNGKGKYILVIYENILKYVNMDSWNRELLEKYCREYSVSIIGFHKANENSLPSTQLKGFPLFLFNNLALKDCFVNPQSPLLHITKAPKVWWGPLPGEDWTIFQYNHSTYQPVLLAELQTEKSLSSLSSKTLFATVILDLGLHDGIQRVLFGNNLNFWLHKLIFLDAISFLSGGRLSLSLDRYILVDIDDIFVGKEGTRMNVEDVKALLETQNKLRTQVTNFTFNLGFSGKFYHTGTDEEDEGDDMLLRHIDEFWWFPHMWSHMQPHLFHNESSLVEQMLLNKQFAKEHGIPTNMGYAVAPHHSGVYPVHIQLYEAWKKVWGIKVTSTEEYPHLKPARYRRGFIHNGIMVLPRQTCGLFTHTIFYKEYPGGPEELDKSIHGGELFQTILLNPISIFMTHLSNYGNDRLALYTFVNLVNFIQSWTNLKLQTLPPVQLAHKYFQLFPEQKDPLWQNPCDDKRHKDIWSREKTCDHLPKFLVIGPQKTGTTALYLFLLMHPSIISNLPSPKTFEEVQFFNGNNYHKGIDWYMDFFPTPSNTTSDFLFEKSANYFHSEEAPRRAAALLPKAKIITILIDPADRAYSWYQHQRSHNDPAALNYSFYEVITTGHWAPKELKTLQNRCLLPGWYATHIERWLTYFHPSQLLIIDGQQLRSDPATVMDEVQKFLGVTPRYNYSQALTFDPQKGFWCQLLEGGKTKCLGKSKGRKYPPMDPESRKFLSRYYRDHNVELSKLLHRLGQPLPSWLRQELQKVR